SEQUENCE LISTING (17 sequences)

SEQ ID NO: 1 - protein, 47 aa, Homo sapiens
DAEFRHDSGYEVHHQKLVFFAEDVGSNKGAIIGLMVGGVVIATVIVI

SEQ ID NO: 2 - DNA, 165 bp, Homo sapiens
ggatccgccaccgacgcagagttccggcacgatagcggctacgaagtgcaccatcagaagctggtcttctttgccgaggacgtgggcagcaacaaaggcgctatcattggactgatggtgggcggggtggtcatcgcaaccgtgatcgtcatttgataactcgag

SEQ ID NO: 3 - DNA, 141 bp, Homo sapiens
gacgcagagttccggcacgatagcggctacgaagtgcaccatcagaagctggtcttctttgccgaggacgtgggcagcaacaaaggcgctatcattggactgatggtgggcggggtggtcatcgcaaccgtgatcgtcatt

SEQ ID NO: 4 - DNA, 12 bp, Homo sapiens
ggatccgccacc

SEQ ID NO: 5 - DNA, 12 bp, Homo sapiens
tgataactcgag

SEQ ID NO: 6 - DNA, 25 bp, Artificial Sequence
  Synthetic
aaaggatccatggatgcagaattcc

SEQ ID NO: 7 - DNA, 25 bp, Artificial Sequence
  Synthetic
gcctctagattacgctatgacaaca

SEQ ID NO: 8 - DNA, 25 bp, Artificial Sequence
  Synthetic
aaaggatccatggatgcagaattcc

SEQ ID NO: 9 - DNA, 25 bp, Artificial Sequence
  Synthetic
gccgtcgactaacgctatgacaaca

SEQ ID NO: 10 - DNA, 35 bp, Artificial Sequence
  Synthetic
tcggaattctgcatctcctcctcccgctatgacaa

SEQ ID NO: 11 - DNA, 25 bp, Artificial Sequence
  Synthetic
cggtgttgtcatagcgggaggagga

SEQ ID NO: 12 - DNA, 25 bp, Artificial Sequence
  Synthetic
gttggatacaggccagactttgttg

SEQ ID NO: 13 - DNA, 22 bp, Artificial Sequence
  Synthetic
tcggtatccggtcggatgggag

SEQ ID NO: 14 - DNA, 22 bp, Artificial Sequence
  Synthetic
ctcccactcccgtggcttctag

SEQ ID NO: 15 - DNA, 24 bp, Artificial Sequence
  Synthetic
gttcacacctcgttgtacaccttg

SEQ ID NO: 16 - DNA, 25 bp, Artificial Sequence
  Synthetic
ccagttttacctggtagaagtgatg

SEQ ID NO: 17 - DNA, 30 bp, Artificial Sequence
  Synthetic
aactcagacgacctgaggtcctggatctgt

(19) United States
(12) Patent Application Publication
(10) Pub. No.: US 2014/0227304 A1
(43) Pub. Date: Aug. 14, 2014

(54) VACCINES FOR PREVENTING AND TREATING ALZHEIMER'S DISEASE

(71) Applicants: Bin Wang, Beijing (CN); Yang Yu, Beijing (CN); Xianzhu Zhu, Lenexa, KS (US); Shuang Wang, Beijing (CN)

(72) Inventors: Bin Wang, Beijing (CN); Yang Yu, Beijing (CN); Xianzhu Zhu, Lenexa, KS (US); Shuang Wang, Beijing (CN)

(21) Appl. No.: 14/233,836

(22) PCT Filed: Jul. 27, 2012
(86) PCT No.: PCT/CN2012/079305

§ 371 Date: Apr. 28, 2014

(30) Foreign Application Priority Data
Jul. 29, 2011 (CN) 201110217429.2

(51) Int. Cl. C07K 14/47

(52) U.S. Cl. CPC C07K 14/4711
USPC 424/185.1

(57) ABSTRACT

Disclosed herein are vaccines comprising an amyloid beta antigen and a nucleic acid encoding the amyloid beta antigen, or a variant thereof. T cell proliferation can be inhibited and iTreg cells can be stimulated in the subject administered the vaccine. Further provided are methods for treating Alzheimer's Disease, methods for reducing or slowing the rate of formation of amyloid beta plaques, methods for treating encephalitis, and methods for of reducing brain swelling. The methods can comprise administering the vaccine to a subject in need thereof.

### CROSS-REFERENCE TO RELATED APPLICATIONS

This application is a continuation-in-part of International Patent Application No. PCT/CN2012/079305, filed Jul. 27, 2012, which claims priority to Chinese Patent Application No. 201110217429, filed Jul. 29, 2011, each of which is incorporated herein by reference in its entirety.

### TECHNICAL FIELD

The present invention relates to vaccines and methods for preventing and treating Alzheimer's disease.

### BACKGROUND

Alzheimer's disease (AD) is a neurodegenerative disease characterized by progressive cognitive dysfunction, massive loss of neurons, and deposition of amyloid plaques and neurofibrillary tangles. The pathological accumulation of amyloid is possibly due to a site-specific abnormal processing of amyloid precursor protein (APP). Previous studies have shown that deposition of amyloid beta protein (Aβ), particularly Aβ40 or Aβ42 forms deposited in amyloid plaques, is one of the hallmarks of the disease and could be triggering a T cell-mediated (auto) immune reaction. A large body of evidence supports the Amyloid Cascade Hypothesis, which states that accumulation of Aβ is the initiating step for the onset of AD. Therefore, most research has focused on Aβ, and many Aβ-related therapeutic strategies have been proposed and developed, including immunotherapy.

After successful experiments in AD model mice, a phase IIa immunotherapy trial in patients with mild to moderate AD showed that about 20% of patients receiving Aβ42 protein vaccine had IgG responses. However, the trial had to be stopped because of meningoencephalitis in 6% of the vaccinated patients. Pathology reports showed that the severe cases of meningoencephalitis were strongly related to brain inflammation caused by vaccine-induced T cell infiltrations.

Several approaches have included the use of truncated versions of Aβ42 that excluded T cell epitopes, such as Aβ1-28, 1-16, 1-14, or 1-9 epitopes, as candidate vaccines. Many of the Aβ immunization studies reported reduced cerebral Aβ levels and/or improved cognition in mice, nonhuman primates, or humans. Alternatively, monoclonal antibodies against Aβ42 have been used in passive vaccination, including Bapineuzumab, Solanezumab and Ponezumab. Although selecting the B cell epitope for vaccines may avoid the T cell response, short peptides do not have the same strong ability to stimulate a high titer of IgG, and therefore need to be modified, which adds to production cost and complexity. Passive immunization with antibody may not stimulate the unwanted T cell response, but antibodies are more expensive than protein or DNA vaccines and do not last very long in vivo. There remains a need for effective vaccines against AD that are economical and effective. There remains a need for effective vaccines that elicit and maintain a high level of antibodies against Aβ antigen, while at the same time prevent T cell responses.

### SUMMARY

The present invention is directed to a vaccine comprising an amyloid beta antigen and a nucleic acid encoding the amyloid beta antigen, or a variant thereof. The amyloid beta antigen may comprise one or more amyloid beta polypeptides in tandem. The amyloid beta polypeptide may comprise an amino acid sequence selected from the group consisting of SEQ ID NO: 1, an amino acid sequence that is 95% identical over the entire length of the amino acid sequence of SEQ ID NO: 1, a fragment of SEQ ID NO: 1 comprising at least 20 amino acids, and an amino acid sequence that is 95% identical to a fragment of SEQ ID NO: 1 comprising at least 20 amino acids. The amyloid beta antigen may comprise two copies of the amyloid beta polypeptide in tandem. The amyloid beta antigen may comprise one copy of the amyloid beta polypeptide. The nucleic acid may encode an amyloid beta antigen variant comprising one copy of the amyloid beta polypeptide. The nucleic acid may encode an amyloid beta antigen variant comprising two copies of the amyloid beta polypeptide in tandem. The amyloid beta polypeptide may be Aβ(42). The nucleic acid may further comprise an expression vector. The expression vector may be operably linked to regulatory elements. The regulatory elements may be functional in a human cell. The expression vector may be a plasmid.

The present invention is further directed to a method for preventing or treating Alzheimer's Disease in a subject, the method comprising administering to the subject a vaccine comprising an amyloid beta antigen and a nucleic acid encoding the amyloid beta antigen, or a variant thereof, as detailed above. T cell proliferation may be suppressed in the subject relative to a control. The level of iTreg cells may be increased relative to a control. Infiltration of CD4+ T cells into the brain may be suppressed in the subject relative to administration of amyloid beta antigen alone. The levels of IFN-γ may be decreased relative to a control, and the levels of Foxp3, IL-10, and TGF-β may be increased relative to a control. The level of IgG may be increased in the subject relative to a reference level. The percent area of the subject's brain comprising amyloid beta plaque may be reduced to 0-0.3%. The method may further comprise testing for spatial learning and memory. Spatial learning and memory may be tested with Morris Water Maze. Spatial learning and memory may be improved by 10-50%.

The present invention is further directed to a method for reducing or slowing the rate of formation of amyloid beta plaques in a subject, the method comprising administering to the subject a vaccine comprising an amyloid beta antigen and a nucleic acid encoding the amyloid beta antigen, or a variant thereof, as detailed above.

The present invention is further directed to a method for treating encephalitis in a subject, the method comprising administering to the subject a vaccine comprising an amyloid beta antigen and a nucleic acid encoding the amyloid beta antigen, or a variant thereof, as detailed above.

The present invention is further directed to a method of reducing brain swelling in a subject, the method comprising administering to the subject a vaccine comprising an amyloid beta antigen and a nucleic acid encoding the amyloid beta antigen, or a variant thereof, as detailed above.

### BRIEF DESCRIPTION OF THE DRAWINGS

FIGS. 1a-b show pMD 18-T-Aβ42 and pVAXI-Aβ42 plasmid enzyme digestion electrophoretograms. FIG. 1a shows BamHI and XbaI double enzyme digestions; Lanes 1,2: pMD18-T-Aβ42, M1: DL2000 marker; M2: DL15000 marker. FIG. 1b shows BamHI and XbaI double enzyme digestions; Lanes 3,4: pVAX1-Aβ42, M: DL15000 marker.

FIG. 2 shows pVAX1-Aβ42 expression identified by RT-PCR.

FIGS. 3a-b show pMD 18-T-Aβ42 and pET28a-Aβ42 plasmid enzyme digestion electrophoretograms. FIG. 3a shows BamHI and SalI double enzyme digestions; Lanes 1,2: pMD18-T-Aβ42, M: DL2000 marker. FIG. 3b shows BamHI and SalI double enzyme digestions; Lanes 3, 4: pET28a-Aβ42, M1: DL2000 marker.

FIGS. 4a-b show pMD 18-T-Aβ422c enzyme digestion electrophoretogram and pET28a-Aβ422c bacterial colony PCR electrophoretogram. FIG. 4a shows BamHI and EcoRI double enzyme digestions; Lanes 1,2: pMD18-T-Aβ42, M: DL2000 marker. FIG. 4b shows pET28a-Aβ422c bacterial colony PCR result (lanes 3-7); Lanes M: DL2000 marker.

FIGS. 5a-c show an SDS-page (FIGS. 5a-5b) and Western Blot (FIG. 5c) of prokaryotic expression of one copy of Aβ42 protein and two copies of proteins.

FIGS. 6a-b show a comparison of anti-Aβ42 antibody IgG of immunized Balb/e and C57 mice.

FIGS. 7a-b show a comparison of T cell proliferation experiment results of immunized Balb/e and C57 mice.

FIGS. 8a-b show proliferation experiment results of T cell and antibodies of old immunized mice.

FIGS. 9a-b show results of Aβ42 antigen co-immunization dosage experiment. FIG. 9a shows the detection results of antibody titers and FIG. 9b shows the T-cell proliferation result by using an MTT method 7 days after boosting immunization.

FIGS. 10a-b show long-term effect experimental results of Aβ42 antigen co-immunization. FIG. 10a shows the level of the anti-Aβ42 antibody IgG detected in serum collected on day 28, day 42 and day 56 after the third immunization. The titers of the mice of the co-immunization group reached the highest level of 640,000 times on day 42, and declined to 128,000 times on day 56. FIG. 10b shows the condition of T-cell proliferation detected in mice sacrificed on day 57.

FIG. 11 shows results of Dot Blot detection for binding capacity of Aβ42 immune antiserum and Aβ protein fiber.

FIG. 12 shows fluorescence staining results of binding of antiserum and A precipitation in the brains of APP/PS1 senile dementia transgenic ill mice. Blue is Dapi staining and shows cells. Red is a result of incubation with serum from different immunization groups, the red spots in the figures are Aβ precipitate. Positive control group: 3552 Aβ monoclonal antibody is stained, and negative control is preimmune serum PT group.

FIGS. 13a-b show cytokine expression results of Aβ42 protein and DNA co-immunization.

FIGS. 14a-d show the condition of CD4+ T cells in the brains of immunized mice showing that positive T cells were not observed in the Aβ protein and DNA-co-immunization group.

FIGS. 15a-h show results of Aβ42 protein and DNA co-immunization of APP senile dementia model mice. FIG. 15a shows a schematic diagram of the water maze experiment. FIG. 15b shows the track chart of mice swimming at day 2 of the water maze experiment. FIG. 15c shows the time statistic chart of mice seeking platform at day 1 to day 5 of the water maze experiment. FIG. 15d shows the time statistic histogram of mice seeking platform at day 2 of the water maze experiment. FIG. 15e shows the track chart of mice swimming at day 6 of the water maze experiment. FIG. 15f shows the time statistic histogram of mice occurring in zone 4 and zone 5 at day 6 of water maze experiment. FIG. 15g shows the results of antibody titer detection. FIG. 15h shows the results of T cell proliferation by MTT method.

FIGS. 16A-C show co-immunization against Aβ induces high levels of anti-Aβ42 IgG. Serum samples were collected 7 days after the last immunization of C57BL/6 mice (A) or APP695 mice (B) and anti-Aβ IgG titer was determined by ELISA. Mean titers (n=6) are expressed as log 10 units. (C) Immunofluorescence staining of amyloid plaques in hippocampal and cortical sections of 9-month-old APPPS1 tg mice with anti-sera from untreated mice (naïve) or co-immunized (Co-Aβ42) or protein immunized (pro-Aβ42) mice. Polyclonal anti-Aβ antibody 3552 served as a positive control. Red: secondary antibody staining; Blue: Dapi staining. All data are presented as mean±SD. Statistical analysis was performed using parametric one-way ANOVA and T-test was used for comparing two groups. *P<0.05. Shown is one of three independent experiments with similar results.

FIGS. 17A-B show co-immunization reduces amyloid-β deposition in brains of APP695 mice. Brain sections were taken from 14-month-old AD mice (n=5) after the fifth immunization and stained with thioflavin S. Cortical sections were viewed under the fluorescent microscope at 488 nm emission (A), and quantification and statistical analysis of plaque counts and plaque area were performed using the IPP program (B). The remaining brain tissues were used to isolate the insoluble protein fraction and subjected to an ELISA assay with a specific monoclonal antibody against Aβ to quantify the Aβ42 protein (C). All data are presented as mean±SD. Statistical analysis was performed using parametric one-way ANOVA and T-test was used for comparing two groups. *p<0.05 compared with APP695 mice.

FIGS. 18A-D show co-immunization improves behaviour of APP695 mice in open-field and water-maze tests. Groups of APP695 mice (n=9) were treated with the designated vaccines. Age- and sex-matched non-transgenic littermates (wild-type) without any treatment were used as a control. APP695 mice without vaccination were used as the AD model disease control. (A) Thigmotaxis was assessed by the amount of time that mice spent in the peripheral and central zones in autonomous behaviors in the open-field test. (B) The frequency of exhibition of total mobile changes in the open-field test. (C) Abilities of learning and memory in the Morris water maze. Plotted is the escape latency during 3 d of hidden platform tests. (D) After withdrawal of the platform on the fourth day the duration time in the original underwater platform area zones 4 and 5 was determined. All data are presented as mean±SD. For the Morris water maze tests, escape latency in the hidden platform trial was analyzed with two-way ANOVA of repeated measurements; one-way ANOVA was used for the data obtained from the probe trial. T-test was used for comparing two groups. *p<0.05, **p<0.01 compared with disease model mice.

FIG. 19 shows co-immunization suppresses the expression of inflammatory cytokines in brains of APP695 tg mice. Protein lysates were prepared from the brains of WT, APP695, and the vaccinated mice 7 d after the fifth immunization (n=5). The expression of cytokines were determined by CBA. Levels of IFN-γ, TNF-α, IL-1β, IL-6 were analyzed and their means±SD are expressed in pg per 100 mg brain tissue (pg/100 mg). Statistical analysis was performed using parametric one-way ANOVA analysis of variance and T-test was used for comparing two groups. *p<0.05 compared with APP695 mice.

FIGS. 20A-C show co-immunization suppresses T-cell proliferation by inducing CD25-iTreg. Splenocytes were isolated from vaccinated and control mice 7 days after the fifth immunization and used for intracellular staining or for proliferation assays (n=6). (A) Frequency of FoxP3⁺ cells in the total CD4⁺ T cells after the co-immunization compared with other groups by FACS analysis. (B) The level of T-cell proliferation was assayed using a MTT assay. The splenocytes were re-stimulated for 3 days in vitro using Aβ42 protein as a specific antigen, BSA as a non-specific antigen, or anti-CD3 as a positive control. To all T cells anti-CD28 was added as a co-stimulant. Proliferation rate was expressed as stimulation index (SI). (C-D) Splenocytes were re-stimulated for 12 hours in vitro using Aβ42 protein as a specific antigen and blocked by BFA for 8 hr. The lymphocytes were first gated on CD4+ T cells and further used to intracellular stain for TGF-β and IL-10, IL-4 and IFN-γ followed by FACS analysis (C). Shown is a result typical of three independent experiments. All data are presented as mean±SD. Statistical analysis was performed using parametric one-way ANOVA analysis of variance and T-test was used for comparing two groups. *p<0.05, **p<0.01 compared with protein-vaccinated model mice.

FIGS. 21A-E show co-immunization prevents the EAE induced by Aβ42 protein. (A) Schematic representation of Aβ42-EAE induction and vaccination. The EAE animal model was induced by immunizing with Aβ42 protein+CFA+PT (Aβ42-EAE model) on days 0, then on day 1 PT was given for the second time. The induced mice were further co-immunized or protein-immunized on days 2 and 14. T cells infiltratering brain were assessed by isolating T cells from the brain on day 21. (B) Single cell suspensions of brains were isolated from EAE induced animals with or without vaccinations or from naïve C57BL/6 mice and stained on day 21. (C) The cells were immune-stained for CD3 and CD4, and analyzed by FACS. The upper left panel shows cells from naïve mice with no antibody staining, followed by antibody-stained cells from the naïve mice, from the MOG-induced EAE model, from the A342-induced EAE model, from the Aβ42 induced EAE mice vaccinated with Aβ42 protein vaccine, or from the co-immunized mice. (D) Number of CD4⁺ T cells in brain. The number of double positive CD3⁺ and CD4⁺ T cells per 10⁶ brain cells from the groups of 6 animals used in panel C were statistically analyzed and plotted. (E) Histopathological examination of brain cortex on the sixth day after EAE model induction, stained with H&E. The naïve section was from naïve mice without any treatment or induction. Shown are results representative of three independent experiments. Statistical analysis was performed using parametric one-way ANOVA analysis of variance and T-test was used for comparing two groups. All data are presented as mean±SD. ****p<0.0001 compared with protein-vaccinated Aβ42-EAE mice.

FIGS. 22A-C show the effects of iTreg transfer on T cell infiltrations in the brain in the Aβ42 induced EAE model. CD4⁺CD25⁻Foxp3⁺ iTregs were isolated from co-immunized Foxp3-eGFP mice on day 7 after the second co-immunization and adoptively transferred into Aβ42-induced-EAE model mice on days 5 and 12 after EAE induction. (A) Schematic representation of iTreg transfer to prevent Aβ42-EAE. iTregs were isolated from Foxp3-eGFP mice after the second co-immunization and adoptively transferred into Aβ42-EAE model on days 5 and 12 after the model was induced. CD4⁺CD25⁺Foxp3⁺nTreg transfers were used as a control. (B) CD3⁺ and CD4⁺ T cells among total brain cells were stained and analyzed on day 21 by FACS. (C) The number of double positive CD3⁺ and CD4⁺ T cells per 10⁶ brain cells from the groups of 6 animals as detected in panel B were statistically analyzed and plotted. Results shown are typical of three independent experiments. All data are presented as mean±SD. Statistical analysis was performed using parametric one-way ANOVA analysis of variance and T-test was used for compared two groups. ***p<0.001 compared with Aβ42-EAE mice.

FIGS. 23A-C show the effects of co-immunization on immune responses. Groups of C57BL/6 mice (n=6) were immunized by the co-immunization with protein:DNA at various ratios (100 μg:100 μg, 100 μg:200 μg, 100 μg:300 μg, and 200 μg:100 μg), protein vaccine alone, DNA vaccine alone, or were un-vaccinated as a control. (A) Total anti-Aβ IgG was analyzed by ELISA on day 7 after the 3rd immunization. Mean titers are expressed as log 10. (B) Splenocytes were isolated from each group on day 7 after the 3rd immunization and stimulated for 3 d in vitro using Aβ42 protein as the specific antigen, BSA as a non-specific antigen, or anti-CD3 as a positive control stimulant; all the T cells were given anti-CD28 as a co-stimulant. The level of T-cell proliferation was evaluated by MTT assay and the data are expressed as a stimulation index. (C) The numbers of CD4+CD25−Foxp3+ iTreg cells as a percentage of total CD4+ T cells was analyzed by FACS analysis. Data shown are the result of three independent experiments. All data are presented as mean±SD. Statistical analysis was performed using parametric one-way ANOVA and T-test was used for comparing two groups. *p<0.05, **p<0.01 compared with Aβ42 protein vaccinated mice.

### DETAILED DESCRIPTION

The current invention relates to the discovery that administration of a vaccine comprising an Aβ antigen and a nucleic acid encoding the Aβ antigen, or variant thereof, induces the production of anti-Aβ antibodies, as indicated by increased levels of IgG against the Aβ antigen, and results in the clearance of amyloid plaques and the improvement in cognitive behavior in AD patients. Strikingly, it was also discovered that the co-immunization of the Aβ antigen and the nucleic acid encoding the Aβ antigen, or variant thereof, induces antigen-specific iTreg cells that ameliorate T cell-mediated brain inflammation. The prevention of inflammation is due, at least in part, to the suppressive effects of iTregs on CD4+ effector T cell function. This is the first demonstration that a co-immunization strategy ameliorates AD pathology without notable adverse effects.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

“Adjuvant” as used herein means any molecule added to the immunotherapeutic composition described herein to enhance the immunogenicity of the antigens.

“Amyloid beta” or “Aβ” as used interchangeably herein refers to an amino acid peptide that is processed from the human amyloid precursor protein (APP). Aβ is a component of amyloid plaques in association with Alzheimer's disease. Aβ is the main component of certain deposits found in the brains of patients with Alzheimer's disease.

“Amyloid beta plaque” or “Aplaque” as used interchangeably herein refers to extracellular deposits of amyloid beta polypeptides in the gray matter of the brain. The formation of amyloid beta is a normal occurrence in the human body. The accumulation of Aβ leads to the aggregation of Aβ and the formation of plaques, which are neurotoxic.

“Amyloid beta polypeptide” as used herein refers to an Aβ(X-Y) peptide.

“Aβ(X-Y) peptide” as used herein refers to the amino acid sequence from amino acid position X to amino acid Y of the human sequence Aβ protein including both X and Y, in particular to the amino acid sequence from amino acid position X to amino acid position Y of the amino acid sequence

(SEQ ID NO: 1)
DAEFRHDSGYEVHHQKLVFFAEDVGSNKGAIIGLMVGGVVIATVIVI

or variants thereof. For example, “Aβ(1-42)” or “Aβ42” as used interchangeably herein refers to the amino acid sequence from amino acid position 1 to amino acid position 42 of the human Aβ protein including both 1 and 42, in particular to the amino acid sequence of SEQ ID NO: 1, or variants thereof, whereas “Aβ(1-40)” or “Aβ40” as used interchangeably herein refers to the amino acid sequence from amino acid position 1 to amino acid position 40 of the human Aβ protein including both 1 and 40, in particular to the amino acid sequence of SEQ ID NO: 1, or variants thereof.

“Amyloid beta antigen” or “Aβ antigen” as used herein refers to Aβ polypeptide capable of eliciting an immune response in a mammal.

“Amyloid precursor protein” or “APP” as used interchangeably herein refers to an integral membrane protein expressed in many tissues and concentrated in the synapses of neurons. APP is the precursor molecule whose proteolysis generates Aβ.

“Control” or “control subject” as used herein refers to a subject who has not been administered the disclosed vaccine. The control subject can be a healthy subject, i.e., a subject having no clinical signs or symptoms of AD, a subject who has been diagnosed with AD but has not been administered the disclosed vaccine, or a subject who has been diagnosed with AD but has been administered an Aβ antigen only vaccine.

“Encephalitis” as used herein refers to an acute inflammation of the brain. Encephalitis with meningitis is known as meningoencephalitis. Symptoms include headache, fever, confusion, drowsiness, and fatigue. More advanced and serious symptoms include seizures or convulsions, tremors, hallucinations, and memory problems.

“Fragment” as used herein with respect to nucleic acid sequences means a nucleic acid sequence or a portion thereof, that encodes a polypeptide capable of eliciting an immune response in a mammal. The fragments can be DNA fragments selected from at least one of the various nucleotide sequences that encode Aβ polypeptides or variants thereof. “Fragment” with respect to polypeptide sequences means a polypeptide capable of eliciting an immune response in a mammal.

“Immune response” as used herein means the activation of a host's immune system, e.g., that of a mammal, in response to the introduction of antigen. The immune response can be in the form of a cellular or humoral response, or both.

“Nucleic acids” as used herein can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide” or “polypeptide” is a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

The term “reference level” or “reference” as used herein refers to the level of IgG levels in a sample group that serves as a reference against which to assess the IgG levels in an individual or sample group. For example, the reference level may be the level of IgG in a control sample from AD patients that have not been treated with the disclosed vaccine.

“Subject” as used herein can mean a mammal that wants to or is in need of being treated with the herein described immunotherapeutic composition. The mammal can be a human, chimpanzee, dog, cat, horse, cow, mouse, or rat.

“Treatment” or “treating,” when referring to protection of an animal from a disease, means preventing, suppressing, repressing, ameliorating, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing or ameliorating the disease involves administering a composition of the present invention to an animal after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; (iv) a nucleic acid that hybridizes under stringent conditions to the referenced nucleic acid, complement thereof, or a sequences substantially identical thereto; or (v) a nucleic acid that encodes more than one copy of the amyloid beta polypeptide, such as two copies in tandem.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can mean a substantially identical sequence. Variant can mean a functional fragment thereof. Variant can also mean multiple copies of a protein. The multiple copies can be in tandem or separated by a linker. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a nucleic acid sequence that is substantially identical over the full length of the full gene sequence or a fragment thereof. The nucleic acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. VACCINE

Provided herein is a vaccine that is comprised of an Aβ antigen and a DNA encoding the Aβ antigen or a variant thereof. The vaccine can induce antigen-specific iTreg cells that inhibit antigen-specific T cell function. The combination of the amyloid beta antigen and DNA encoding the amyloid beta antigen or variant thereof in the vaccine induces iTreg cells efficiently against Aβ far better than either a vaccine comprising an Aβ antigen or its corresponding DNA alone. The vaccine induces high titers of antibody against Aβ, which effectively reduced plaque formation in AD mouse models. The vaccine also induces iTregs and strongly reduces brain inflammation and infiltration of T cells into the brain. The vaccine ameliorates AD development.

The Aβ antigen can induce antigen-specific iTreg cells that inhibit antigen-specific T cell function. Co-immunization with Aβ protein antigens and nucleic acids encoding said Aβ antigens or variants thereof can induce regulatory DCs (DCregs) of a CD11c⁺CD40^{low}IL-10⁺ phenotype in vitro and in vivo, which in turn can mediate antigen-specific tolerance.

Conventional dendritic cells (DCs) are specialized antigen-presenting cells (APCs) that can be broadly classified into the CD11c⁺CD8a⁺ and CD11c⁺CD8a⁻ subtypes, both of which have a remarkable functional plasticity in the induction of immunity or tolerance, depending on their maturation status Immature DCs (iDCs) can promote tolerance by converting naïve T cells into the CD4⁺Foxp3⁺ regulatory T cells (Tregs). Signals form the nucleic acid construct and the protein of the vaccine can act in a concerted manner to activate regulatory signals that convert normal DCs into DCregs.

DNA and protein antigen co-immunization induces DCregs by allowing co-uptake of the nucleic acid and protein immunogens by the same DC primarily via caveolae-mediated endocytosis. This event down-regulates the phosphorylation of Cav-1 and up-regulates Tollip, which in turn initiates downstream signaling that up-regulates SOCS1 and down-regulates NF-κB and STAT-1α. The down-regulation of NF-κB explains the CD40low and IL-10+ phenotype of the co-immunization-induced DCregs. DCregs can be generated in vitro in both primary DCs and DC lines by feeding them with DNA and protein immunogen for as short as 24 h. The in vitro generated DCregs are effective for treating inflammatory and autoimmune diseases, presumably by inducing antigen-specific CD25− iTreg.

DNA and protein antigen co-immunization induces CD40low IL-10high tolerogenic DCs, which in turn stimulates the expansion of antigen specific CD25− iTreg (CD4+CD25−Foxp3+ regulatory T cells). More highly antigenic epitopes stimulate more efficient induction of CD25− iTreg. This positive effect of strong antigen stimulation on efficient induction of CD25− iTreg was supported by the observation that induction of CD25− iTreg by tolerogenic DC can be blocked by anti-MHC-II antibodies, the number and the suppressive activity of CD25− iTreg correlates positively with the antigenicity of an epitope to activate T cells, and CD25− iTreg cells induced by a more antigenic epitope are more effective in preventing the development of an allergic condition. Multiple copies of the Aβ antigen, as detailed below, can provide a stronger antigenic epitope and more efficiently induce CD25− iTreg. Strong antigenicity of an epitope may be predicted from the affinity of the epitope to MHC. With more efficient CD25− iTreg induction, a subject would be more effectively protected from and treated for allergenic reaction to the antigen.

Cav-1 is the key protein to form caveolae. It also regulates signal transduction through compartmentalization of numerous signaling molecules. Cav-1, Tollip, and IRAK-1 form a complex to suppress the IRAK-1's kinase activity during resting conditions. Cav-1 dissociates from the complex once phosphorylated, which leads to phosphorylation of IRAK-1 in the cytosol and activation of the downstream signaling cascade, including translocation of NF-κB25. Co-uptake of nucleic acid and protein down-regulates phosphorylation of Cav-1, thereby preventing the activation NF-κB. Accordingly, a nucleic acid antigen and a protein antigen can convert normal DCs into DCregs. The same DC is required for acquisition of the DCreg phenotype and function and that the co-uptake event triggers Cav-1 ant Tollip co-dependent signaling that up-regulates SOCS1 and down-regulates NF-κB and STAT-1α.

iTreg cells cause a reduction in inflammatory T_{Helper} and Tₖᵢₗₗₑᵣ cells. The iTreg suppression can occur by interaction with the antigen-presenting cells, including DCs and epithelial cells, for example in the lung or other organ, where the antigen specific iTreg cells are retained by reducing their expression of the egress molecule S1P1. The interaction upregulates expression of chemoattracting IP-10 of antigen specific APCs, which trap the CXCR3⁺ inflammatory T cells into epithelial cells (i.e. T_{H1}, T_{K1}, etc.). Twenty percent of these trapped T cells undergo apoptosis and a few are then converted into IL10 and TGF-beta expressing Treg cells. Therefore, the inflammatory T cells are reduced in organs, like the lungs, and conditions, such as asthma, are ameliorated.

a. Aβ Antigen

The Aβ antigen can comprise an Aβ polypeptide that is capable of eliciting an immune response in a mammal against Aβ. The Aβ antigen can comprise a polypeptide as detailed below. The Aβ antigen can comprise the full length Aβ polypeptide of SEQ ID NO: 1, a variant thereof, a fragment thereof, or a combination thereof. The Aβ antigen can comprise Aβ polypeptide having an amino acid sequence that is 80%, 81%, 82%, 83%, 84%, 85%, 86%, 87%, 88%, 89%, 90%, 91%, 92%, 93%, 94%, 95%, 96%, 97%, 98%, or 99% identical over the entire length of the amino acid sequence of SEQ ID NO: 1. The Aβ antigen can comprise a fragment of SEQ ID NO: 1 comprising at least 20 amino acids. The Aβ antigen can comprise Aβ polypeptide having an amino acid sequence that is 80%, 81%, 82%, 83%, 84%, 85%, 86%, 87%, 88%, 89%, 90%, 91%, 92%, 93%, 94%, 95%, 96%, 97%, 98%, or 99% identical to a fragment of SEQ ID NO: 1 comprising at least 20 amino acids.

The Aβ antigen can comprise an Aβ(X-Y) peptide of SEQ ID NO: 1, wherein the amino acid sequence from amino acid position X to amino acid Y of the human sequence Aβ protein including both X and Y, in particular to the amino acid sequence from amino acid position X to amino acid position Y of the amino acid sequence DAEFRHDSGYEVHHQKLVFFAEDVGSNKGAIIGLMVGGVVIATVIVI (corresponding to amino acid positions 1 to 47; the human query sequence; SEQ ID NO: 1) or variants thereof. The Aβ antigen can comprise an Aβ polypeptide of Aβ(X-Y) polypeptide wherein X can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 and Y can be 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, or 15. The Aβ polypeptide can comprise a fragment that is at least 15, at least 16, at least 17, at least 18, at least 19, at least 20, at least 21, at least 22, at least 23, at least 24, at least 25, at least 30, at least 35, at least 36, at least 37, at least 38, at least 39, at least 40, at least 41, at least 42, at least 43, at least 44, at least 45, or at least 46 amino acids of SEQ ID NO: 1. The Aβ polypeptide can be Aβ42. The Aβ antigen can comprise at least one copy, at least two copies, at least three copies, at least four copies, or at least five copies of the Aβ polypeptide.

Immunogenic fragments of SEQ ID NO: 1 can be provided Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:1. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences similar to immunogenic fragments of SEQ ID NO:1 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% identical to SEQ ID NO:1. Some embodiments relate to immunogenic fragments that have 96% identity to the immunogenic fragments of consensus protein sequences herein. Some embodiments relate to immunogenic fragments that have 97% identity to the immunogenic fragments of consensus protein sequences herein. Some embodiments relate to immunogenic fragments that have 98% identity to the immunogenic fragments of consensus protein sequences herein. Some embodiments relate to immunogenic fragments that have 99% identity to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:1. Immunogenic fragments can be at least 95%, at least 96%, at least 97% at least 98% or at least 99% identical to fragments of SEQ ID NO: 1. In some embodiments, immunogenic fragments include sequences that encode a leader sequence, such as for example an immunoglobulin leader, such as the IgE leader. In some embodiments, fragments are free of coding sequences that encode a leader sequence.

b. Nucleic Acid

Further provided herein is a nucleic acid that encodes the Aβ antigen or variant thereof. The nucleic acid can include an encoding sequence that encodes the Aβ antigen or variant thereof. The nucleic acid can also include additional sequences that encode linker or tag sequences that are linked to the antigen by a peptide bond. The Aβ antigen and the Aβ antigen encoded by the nucleic acid can be the same or different Aβ polypeptide. The nucleic acid may include the polynucleotide sequence GACGCAGAGTTCCGGCACGATAGCGGCTACGAAGTGCACCATCAGAAGCTGGTC TTCTTTGCCGAGGACGTGGGCAGCAACAAAGGCGCTATCATTGGACTGATGGTG GGCGGGGTGGTCATCGCAACCGTGATCGTCATT (SEQ ID NO: 3). The nucleic acid may include the polynucleotide sequence GGATCCGCCACCGACGCAGAGTTCCGGCACGATAGCGGCTACGAAGTGCACCAT CAGAAGCTGGTCTTCTTTGCCGAGGACGTGGGCAGCAACAAAGGCGCTATCATT GGACTGATGGTGGGCGGGGTGGTCATCGCAACCGTGATCGTCATTTGATAACTCG AG (SEQ ID NO: 2). The nucleic acid may include optional sequences such as those underlined in SEQ ID NO: 2 (5′-end GGATCCGCCACC, SEQ ID NO: 4; and 3′-end TGATAACTCGAG, SEQ ID NO: 5), which can enhance expression.

The nucleic acid can encode a variant of the Aβ antigen. The variant can include more than one copy of the amyloid beta polypeptide, such as two copies in tandem. The vaccine can include an Aβ antigen that comprises two copies of the Aβ polypeptide in tandem with a nucleic acid encoding an Aβ antigen variant that comprises one copy of the Aβ polypeptide. The vaccine can include an Aβ antigen that comprises one copy of the Aβ polypeptide with a nucleic acid encoding an Aβ antigen variant that comprises two copies of the Aβ polypeptide. The vaccine can include an Aβ antigen that comprises two copies of the Aβ polypeptide in tandem with a nucleic acid encoding an Aβ antigen variant that comprises two copies of the Aβ polypeptide.

c. Vector

Further provided herein is a vector that includes the nucleic acid as detailed above. The vector can be capable of expressing the antigen. The vector can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors can have expression signals such as a strong promoter, a strong termination codon, adjustment of the distance between the promoter and the cloned gene, and the insertion of a transcription termination sequence and a PTIS (portable translation initiation sequence).

i. Expression Vectors

The vector can be circular plasmid or a linear nucleic acid vaccine. The circular plasmid and linear nucleic acid are capable of directing expression of a particular nucleotide sequence in an appropriate subject cell. The vector can have a promoter operably linked to the antigen-encoding nucleotide sequence, which can be operably linked to termination signals. The vector can also contain sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter which initiates transcription only when the host cell is exposed to some particular external stimulus. In the case of a multicellular organism, the promoter can also be specific to a particular tissue or organ or stage of development.

ii. Circular and Linear Vectors

The vector can be circular plasmid, which can transform a target cell by integration into the cellular genome or exist extrachromosomally (e.g. autonomous replicating plasmid with an origin of replication).

The vector can be pVAX, or pET, or any other expression vector capable of expressing the DNA and enabling a cell to translate the sequence to an antigen that is recognized by the immune system.

iii. Promoter, Intron, Stop Codon, and Polyadenylation Signal

The vector can include a promoter. A promoter can be any promoter that is capable of driving gene expression and regulating expression of the isolated nucleic acid. Such a promoter can be a cis-acting sequence element required for transcription via a DNA dependent RNA polymerase, which transcribes the antigen sequence described herein. Selection of the promoter used to direct expression of a heterologous nucleic acid depends on the particular application. The promoter can be positioned about the same distance from the transcription start in the vector as it is from the transcription start site in its natural setting. However, variation in this distance can be accommodated without loss of promoter function.

The promoter can be operably linked to the nucleic acid sequence encoding the antigen and signals required for efficient polyadenylation of the transcript, ribosome binding sites, and translation termination. The promoter can be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector can include an enhancer and an intron with functional splice donor and acceptor sites. The vector can contain a transcription termination region downstream of the structural gene to provide for efficient termination. The termination region can be obtained from the same gene as the promoter sequence or can be obtained from different genes.

d. Other Components of Vaccine-Adjuvants, Excipients

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. In some embodiments, the transfection facilitating agent is poly-L-glutamate, and the poly-L-glutamate can be present in the vaccine at a concentration less than 6 mg/mL. The transfection facilitating agent can also include surface active agents such as immune-stimulating complexes (ISCOMS), Freunds incomplete adjuvant, LPS analog including monophosphoryl lipid A, muramyl peptides, quinone analogs, and vesicles such as squalene and squalene, and hyaluronic acid can also be used administered in conjunction with the genetic construct. The plasmid vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent can be a polyanion or polycation such as poly-L-glutamate (LGS), or a lipid. Concentration of the transfection agent in the vaccine can be less than 4 mg/mL, less than 2 mg/mL, less than 1 mg/mL, less than 0.750 mg/mL, less than 0.500 mg/mL, less than 0.250 mg/mL, less than 0.100 mg/mL, less than 0.050 mg/mL, or less than 0.010 mg/mL.

The pharmaceutically acceptable excipient can be an adjuvant. The adjuvant can be other genes that are expressed in alternative plasmid or are delivered as proteins in combination with the plasmid above in the vaccine. The adjuvant can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, and CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful adjuvants include those encoding: MCP-1, MIP-1α, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRCS, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2, and functional fragments thereof.

The vaccine can further comprise a genetic vaccine facilitator agent as described in U.S. Serial No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 3. Methods of Using the Vaccines

The present invention is directed to a method of treating or preventing AD or a symptom related to AD in a subject using the vaccine. AD is a neurodegenerative disease characterized by progressive cognitive dysfunction, massive loss of neurons, and deposition of amyloid plaques and neurofibrillary tangles. AD can be diagnosed by tests that evaluate behavior and thinking abilities (see e.g., Alzheimer's Criteria described in Diagnostic and Statistical Manual of Mental Disorders published by the American Psychiatric Association) and/or a brain scan using computed tomography, magnetic resonance imaging, single-photon emission computed tomography or positron emission tomography. Symptoms of AD include difficulty in remembering recent events, confusion, irritability, aggression, mood swings, trouble with language, and long-term memory loss. The vaccine can also be used in dementia related cases. The vaccine can also be used to reduce or slow the rate of Aβ plaque, treat encephalitis, or reduce brain inflammation or brain swelling in a subject.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

a. Methods of Treating and/or Preventing AD

The present invention is also directed to methods for treating and/or preventing AD in a subject using a vaccine administered to a subject. The method includes administering to the subject the vaccine. The administration of the vaccine can cause T cell proliferation to be suppressed in the subject. The T cell proliferation can be suppressed by at least about 1-fold, at least about 2-fold, at least about 3-fold, at least about 4-fold, or at least about 5-fold in the subject relative to a control.

The administration of the vaccine can cause the level of iTreg cells to be increased in the subject. The level of iTreg cells can be increased relative to a control.

The administration of the vaccine can cause the infiltration of CD4+ T cells into the brain to be suppressed in the subject. The infiltration of CD4+ T cells into the brain can be suppressed relative to a control.

The administration of the vaccine can cause the levels of IFN-γ to be decreased and the levels of Foxp3, IL-10, and TGF-β to be increased in the subject relative to a control.

The administration of the vaccine can cause the level of IgG to be increased in the subject. The level of IgG can be increased by at least 1-fold, at least about 2-fold, at least about 3-fold, at least about 4-fold, or at least about 5-fold in the subject relative to a reference level.

The administration of the vaccine can decrease the percent area of the subject's brain comprising amyloid beta plaque. The percent area of the subject's brain comprising amyloid beta plaque can be decreased by 10%, 15%, 20%, 25%, 30%, 35%, 40%, 45%, 50%, 55%, 60%, 65%, 70%, 75%, 80%, 85%, 90%, or 95% relative to the subject prior to treatment. The percent area of the subject's brain comprising amyloid beta plaque can be decreased to 0%, 0.05%, 0.10%, 0.15%, 0.20%, 0.25%, 0.30%, 0.35%, 0.40%, 0.45%, or 0.50%.

The treatment of the subject may be monitored by testing for spatial learning and memory. Spatial learning and memory may be tested before, during, or after treatment, or a combination thereof. Spatial learning and memory may be tested with a variety of means known in the art, such as, for example, the Morris Water Maze. In some embodiments, the administration of vaccine improves spatial learning and memory by 10%, 15%, 20%, 25%, 30%, 35%, 40%, 45%, or 50%, relative to the subject prior to treatment.

b. Methods of Slowing or Reducing Formation of Aβ Plaques

The present invention is also directed to methods of reducing or slowing the rate of formation of Aβ plaques in a subject. The method includes administering to the subject the vaccine. The administration of the vaccine can reduce the formation of Aβ plaques in the subject relative to a control.

c. Methods of Treating Encephalitis

The present invention is also directed to methods for treating encephalitis in a subject. The method includes administering to the subject the vaccine.

d. Methods of Reducing Brain Swelling

The present invention is also directed to methods for reducing brain inflammation or brain swelling in a subject. The method includes administering to the subject the vaccine.

### 4. ADMINISTRATION

The vaccine as detailed above can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce iTreg responses. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The nucleic acid of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccines can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup, or elixir. The vaccine can also be a preparation for parenteral, subcutaneous, intradermal, intramuscular, or intravenous administration (e.g., injectable administration), such as a sterile suspension or emulsion.

The vaccine can be incorporated into liposomes, microspheres, or other polymer matrices (such as by a method described in Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation can be carried out via a minimally invasive device.

### 5. KIT

Provided herein is a kit, which may be used for vaccinating a subject. The kit may comprise an Aβ antigen and a nucleic acid encoding the Aβ antigen or variant thereof. The kit may also comprise one or more containers, such as vials or bottles, with each container containing a separate reagent. The kit may further comprise written instructions, which may describe how to use the kit.

The present invention has multiple aspects, illustrated by the following non-limiting examples.

### 6. EXAMPLES

The foregoing can be better understood by reference to the following examples, which are presented for purposes of illustration and are not intended to limit the scope of the invention.

### Example 1

### Materials and Methods

Preparation of DNA.

Plasmid DNA was extracted from Escherichia coli, protein was removed in a phenol-chloroform solution, and double-strand DNA was separated through ethanol precipitation.

Detailed descriptions of the above extracting method and technology can be found in Sambrook et al (ed.), Molecular Cloning (2nd edition 1998, Cold Spring Harbor Laboratory Press, New York), and Li Chaolong et al (ed.), Biological Chemistry and Molecular Biology Experimental Technology, Zhejiang University Press.

Preparation of Protein and Polypeptide.

The protein and the polypeptide were extracted from genetic engineering expression bacteria or cells. The methods are well known and described in detail in Doonan, Protein Purification Protocols (1996, Humana Press, NJ).

Animals.

Animal models have contributed significantly to understanding the underlying mechanisms of AD and for testing potential therapies. The mouse and human genomes are about 85 percent the same, and those similarities have made the mouse a powerful model for studying human biology and disease. Mouse models, such as those described herein, continue to provide reliable means for testing pharmacodynamic properties of candidate molecules on drug targets that may be involved in AD pathogenesis. For example, relationships in animal models can be explored between peripheral biomarkers and readily available neuropathology; these can be translated into human studies where biomarkers are accessible but neuropathology is often not (Sabbagh et al. Am. J. Neurodegener. Dis. 2013, 2, 108-120). Furthermore, results from a Morris Water Maze with mice to test spatial learning and memory can be correlated with human analog studies of the Morris Water Maze (Fitting et al. Spatial Cognition V, 2007, LNAI 4387, 59-75).

Adult female C57/B6 mice (6-8 weeks of age) were purchased from the Animal Institute of the Chinese Medical Academy (Beijing, China). APP695 mice (10-month-old, C57BL/6 background) were obtained from Chinese Academy of Medical Sciences & Comparative Medical Center. Foxp3-eGFP mice (C57BL/6 background) were kindly provided by Dr. Minghui Zhang (Tsinghua University, Beijing, China). APPPS1 mice (C57BL/6 background) were kindly provided by Mathias Jucker, Hertie Institute Tubingen, Germany. All mouse protocols were approved by the Animal Welfare Committees of China Agricultural University, Fudan University and Institute for Age Research. All animals were maintained under specific pathogen-free conditions with 12 hr light-dark cycle.

Reagents.

Recombinant eukaryotic and prokaryotic plasmids were identified by restriction digestion and verified by sequencing analysis. The inserts were sub-cloned into either pVAX1 or pET28a vector (Invitrogen, Carlsbad, Calif.). Expression was analyzed by Western-blot 48 hr after transfection of BHK cells for inserts in the pVAX backbone, or 8 hr after induction of transfected Escherichia coli with 0.1 mM IPTG for inserts in pET28.

Immunization.

Female C57/B6 mice (6-8 weeks old) and APP695 mice (both male and female, 10 months old) were immunized with various regimens via the tibialis anterior muscle on days 0, 14, 28, and 70. These regimens were as follows: Aβ42 protein, 200 μg/mouse; pVAX-Aβ42, 100 μg/mouse; co-immunization, a mixture of 200 μg Aβ42 protein+100 μg pVAX-Aβ42; a positive control in which mice were first immunized with 200 μg Aβ42 protein emulsified with CFA (Sigma-Aldrich, St. Louis, Mo.) then 2nd and 3rd immunizations were with 200 μg Aβ42 protein in IFA (Sigma-Aldrich).

Flow Cytometry.

T cells were isolated from spleens of immunized C57/B6 mice or APP695 transgenic mice on day 7 after the fourth immunization. For intracellular staining, T cells were stimulated with Aβ42 protein at 10 μg/mL for 8 hr and subsequently treated with BFA (BD Biosciences, San Diego, Calif.) for 2 hr in vitro. The cells were blocked with Fc Blocker (BD Biosciences) in PBS for 30 min at 4° C. before being fixed with 4% paraformaldehyde and permeabilized with saponin (Sigma-Aldrich). The splenocytes were intracellularly stained with the appropriate concentrations of PE-labeled antibodies including anti-Foxp3, anti-IL-10, anti-TGF-β, anti-IFN-γ, anti-IL-4, and PE-cy5.5-labeled anti-CD25 antibody (eBioscience, San Diego, Calif.) and FITC-labeled anti-CD4 antibody (eBioscience) for 30 min at 4° C. The cells were washed and analyzed with a FACS Calibur equipped with CellQuest Pro software (BD Biosciences).

Immunofluorescence Staining.

For Aβ-plaque immunohistochemistry, frozen brain sections of 9-month-old APPPS1 transgenic mice were warmed to room temperature for 30 min and fixed in ice cold acetone for 5 min before air drying for 30 min. Sections were incubated with normal serum for 30 min to block non-specific binding of immunoglobulin. The tissue sections were reacted first for 1 hr at room temperature or overnight at 4° C. with anti-sera induced by co-immunization or by protein vaccination or with sera from untreated mice (all sera were at 1:200 dilution), or with monoclonal antibody 3552 (1:2000 dilution) as a positive control. All serum dilutions were made in primary antibody dilution buffer. After incubation the sections were washed 3 times with PBS, then incubated with Alexa Fluor 555 labeled secondary antibody (1:1000 dilution, Invitrogen) for 30 minutes at room temperature and washed 3 times with PBS. After brush transfer of sections onto glass slides, plaques were detected by fluorescence microscopy (Carl Zeiss Jena, Germany) at 546 nm emission. Nuclei were DAPI-stained and detected at 405 nm emission.

T Cell Proliferative Response.

Single-lymphocyte suspensions were obtained from spleens of immunized C57/B6 or APP695 mice on day 7 after the fourth immunization. Cells in RPMI/10 medium (GIBCO, Eggenstein, Germany) containing 10% fetal bovine serum (FBS) were used to determine the T cell-proliferative response by MTT staining after Aβ42 stimulation in vitro for 72 hr. MTT-stained cells were analyzed by an ELISA plate reader (Magellan; Tecan Austria, Grodig, Austria) at 450 nm absorbance.

Measurement of Aβ42 Protein-Specific Antibodies.

The level of anti-Aβ42 serum IgG antibodies was determined by enzyme-linked immunosorbent assay (ELISA) in 96-well plates coated with Aβ42 protein at 10 μg/mL and detected with a secondary goat anti-mouse IgG antibody conjugated with horseradish peroxidase (Bio-Rad, Hercules, Calif.). Absorbance at 450 nm was measured with an ELISA plate reader (Magellan).

Fibrillar Aβ Deposition and Histological Analysis.

Thioflavine-S staining was used, which is commonly used for fluorescent staining of fibrillar amyloid. Brain tissue was fixed in 4% neutral buffered formalin and mounted in paraffin blocks. After deparaffinisation and hydration, the sections were washed in PBS and incubated in a solution containing 0.25% potassium permanganate and 1% oxalic acid until they appeared white. The sections were then washed in water and stained for 3 min with a solution of 0.015% Thioflavin-S in 50% ethanol. Finally, the sections were washed in 50% ethanol and in water, then dried and dipped in Histo-Clear before being cover-slipped with Permount. The sections were examined using three equidistant sections per animal at 488 nm emission. Quantification and statistical analysis of staining was by Image-Pro Plus version 6.0 software (Media Cybernetics, MD, USA)

Behavioral Tests.

A. Open-Field Test

Behaviors of APP695 mice were measured by the open-field test. Ethovision XT monitoring and analysis software (Noldus Company, Netherlands) was used with a 50×50 cm open field. The open field was divided into three areas, which included the peripheral zone (Zone 1) and the central zone (Zones 2+3). Zone 1 was 8 cm away from the edge of the open field. Zone 3 occupied the center area, which was 16% of the total. The remaining area was designated as Zone 2. Each mouse had 5 min of free movement in the open field. The time that each mouse stayed in each zone, as well as the frequency that the mouse was in the state of being mobile, was recorded. Mobility is the state variable including three different variables: immobile, mobile, or highly mobile. The mouse was considered as immobile when the change in area of the mouse between current sample and previous sample (referred to as changed area) was smaller than 20%, as highly mobile when the changed area was larger than 60%, and as mobile when the changed area was between 20% and 60%. The amount of time spent in the peripheral zone is a manifestation of thigmotaxis. Room temperature was constant, and the light level was even across the open field. The open field was wiped clean with 75% alcohol and dried before each experiment to remove residual odors.

B. Morris Water Maze (MWM)

Learning and memory was tested using the Morris water maze, one day after the end of the open-field test. The protocol for the MWM test was modified from previously reported methods. Briefly, the apparatus included a pool with a diameter of 100 cm that was filled with opaque water at 20±1° C. An escape platform (15 cm in diameter) was placed 0.5 cm below the water surface. Geometric objects with contrasting colors were set at the remote ends of the water tank as references. Room temperature was constant at 20±1° C., and the lighting was even throughout the room. Spatial memory was assessed by recording the latency time for the animal to escape from the water onto a submerged escape platform during the learning phase. The platform was in Zone 4 and defined as Zone 5. The mice were subjected to four trials per day for 3 consecutive days. The mice were allowed to stay on the platform for 15 sec before and after each trial. The time that it took for an animal to reach the platform (latency period) was recorded. Twenty-four hours after the learning phase, the mice swam freely in the water tank without the platform for 60 sec, and the time spent in the region was recorded. Monitoring was performed with a video tracking system (Noldus Ltd, Ethovision XT, Holland).

ELISA Detection for Insoluble Aβ42.

Brains of APP695 mice were extracted in Tris-HCl pH 8.0 buffer and centrifuged at 4° C. and 20,000 g for 20 min. After discarding the supernatant, the pellet was washed in Tris-HCl pH 8.0 buffer 3 times and homogenized in 6 M guanidine-Tris buffer. After sonication at room temperature for 30 sec, the sample was incubated for 1 hr at room temperature and then centrifuged at 4° C. and 20,000 g for 20 min. Samples were diluted 1:12 in ELISA dilution buffer (bicarbonate buffer, pH 9.6), incubated in 96-well plates at 4° C. overnight, and assayed with a primary anti-β Amyloid antibody (2B9, diluted at 1:500, Santa Cruz Biotechnology, Texas, USA) and secondary goat anti-mouse IgG antibody conjugated with horseradish peroxidase (diluted at 1:1000, Bio-Rad). Absorbance at 450 nm was measured with an ELISA plate reader (Magellan).

Analysis of Inflammation Cytokines.

Brain tissues were sequentially homogenized in cold PBS after perfusion. Supernatants after centrifugation at 8000 rpm/min at 4° C. for 30 min were diluted 1:10 with cold PBS and assayed by CBA (Cytometric Beads Array, BD Biosciences) to detect expression levels for IL-6, IL-1β, TNF-α, and IFN-γ.

Induction of Experimental Autoimmune Encephalomyelitis (EAE)

Myelin oligodendrocyte glycoprotein induced model of EAE (MOG-EAE)

Female 8-week old C57BL/6 mice were immunized with 200 μg/100 μL MOG35-55 peptide emulsified in 100 μL of 5× complete Freund's adjuvant (CFA; Sigma-Aldrich). Animals were injected with a total of 200 μL of CFA adjuvant, divided between 2 sub-cutaneous posterior auricular sites. Pertussis toxin (200 ng/mouse; Sigma-Aldrich) was given i.p. on days 0 and 1.

Aβ42-EAE Induced Brain Inflammation

To mimic the results obtained from AN-1792 vaccine-induced brain inflammation and encephalitis, Aβ42 protein (200 μg/100 μL) was used to replace MOG in the above MOG-EAE model and this Aβ42-EAE model was used as the positive experimental control. All the induction protocols used the above MOG-EAE model except that 200 μg/100 μL of Aβ42 protein emulsified in 100 μL of 5× complete Freund's adjuvant (CFA; Sigma-Aldrich) was used instead of MOG.

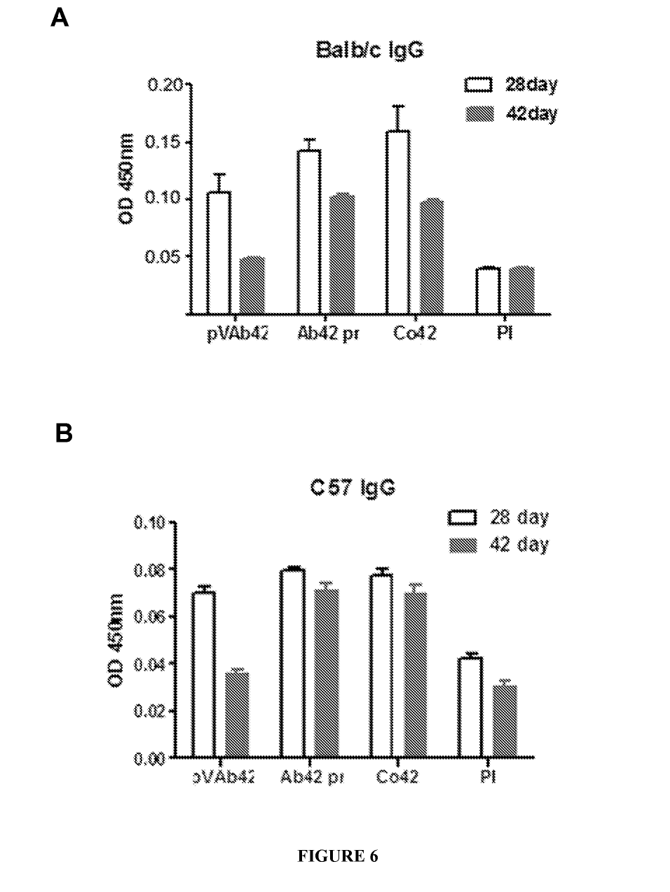

After EAE induction, mice were immunized under various regimens via the tibialis anterior muscle on days 0 and 14 using the protocol shown in FIG. 6A. These regimens were used as follows: the protein group was immunized with Aβ42 protein at 200 μg/mouse, the plasmid group was immunized with pVAX-Aβ42 at 100 μg/mouse, the co-immunization group was immunized with a mixture of 200 μg Aβ42 protein+100 μg pVAX-Aβ42. Positive controls were provided by the untreated MOG-EAE or Aβ42-EAE model. On day 21, the mice were anesthetized before heart perfusion with cold PBS. Brain tissues were isolated and digested with trypsin (MACGENE, Beijing, China) at 37° C. for 0.5 hr to obtain single cell suspensions. Brain cells were stained with FITC-anti-CD3 and APC-anti-CD4 (eBioscience).

CD25-iTreg transfer assay. Foxp3-eGFP mice were used as donor mice and co-immunized intramuscularly with mixture of 200 μg Aβ42 protein+100 μg pVAX-Aβ42 twice with a 14-d interval. Splenocytes were isolated from the co-immunized donor mice 7 d after the 2nd immunization, stained with APC anti-mouse CD4+ and Foxp3+ and PE-cy5-labeled anti-mouse CD25+ antibodies, and sorted using a BD Aria II flow cytometer. Any cells stained for CD25+ were excluded. The sorted CD25−iTreg cells were adoptively transferred intravenously at 1×10⁶ per animal into C57/B6 recipients that had been immunized twice with CFA emulsified Aβ42 protein to induce Aβ42-EAE inflammation.

On day 21, all recipients were anesthetized and heart perfusions were performed with cold PBS. Brains were separated and digested with trypsin at 37° C. for 0.5 hr to obtain single cell suspensions as descripted as above. All cells were stained with FITC-anti-CD3 and APC-anti-CD4.

Statistical Analysis.

Results are presented as means±SD. Except for data from the water-maze experiments, statistical analysis was by parametric one-way ANOVA and T-test was used for comparisons between two groups. For the Morris water maze tests, escape latency in the hidden platform trial was analyzed with two-way ANOVA of repeated measures and one-way ANOVA was conducted on the data obtained from the probe trial. Differences were considered to be statistically significant at p<0.05.

### Example 2

### Preparation of Protein and Polypeptide

Construction of Senile Dementia Aβ42 Eukaryotic Expression Plasmid.

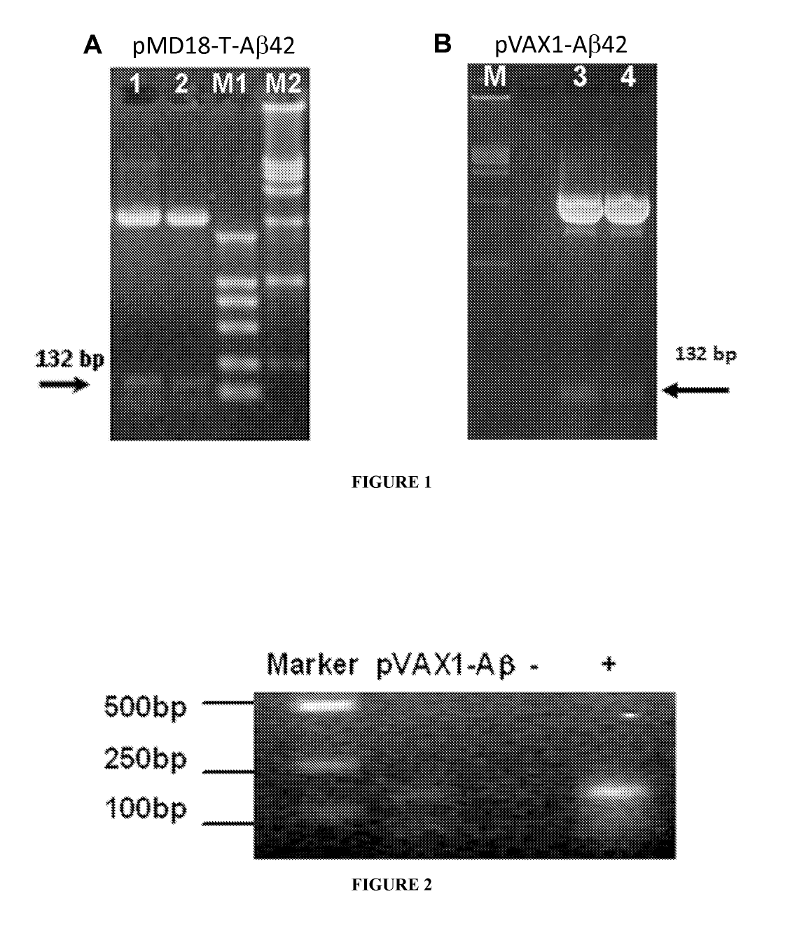

An Aβ42 gene was amplified through PCR under the guidance of a primer P1: 5′-AAAGGATCCATGGATGCAGAATTCC-3′ (SEQ ID NO: 6) and a primer P2: 5′-GCCTCTAGATTACGCTATGACAACA-3′ (SEQ ID NO: 7)(BamH1 and Xbal recognition sites were respectively introduced to the primers 1 and 2) by using a plasmid Aβ42-C3d3 (provided by PHD Mike Argejia) as a template. A reaction system was as follows: 1 μL of plasmid template, 10 pmol of primers 1 and 2, respectively, 500 mM of KCl, 100 mM of Tris-HCl (pH 8.4), 1.5 mM of MgCl₂, 100 μg/mL of BSA, 1 mM of dNTPs, and 2.5 U of Taq DNA polymerase, with the total volume of 25 μL. The reaction conditions were as follows: denaturing for 30 seconds at 94° C., annealing for 30 seconds at 60° C., extending for 30 seconds at 72° C., and conducting total 30 cycles. A DNA amplified fragment in 1.5% agarose gel electrophoresis was recycled and ligated to a pMD18-T cloning vector. A ligated product was transformed into DH5α bacterial competent cells. Positive bacterial colonies were screened through an Ampicillin antibiotic LB solid culture medium. Plasmids were extracted and identified by Bam H1 and Xbal double enzyme digestions, and a result is shown in FIG. 1a. The arrow points out a target fragment ligated to the vector and having the size of 132 bp. A target gene was subjected to BamH1 and Xbal double enzyme digestion. DNA Aβ42 gene fragments in the agarose gel electrophoresis were recycled and ligated to a pVAXI (Invitrogen) eukaryotic expression vector. The ligated product was transformed to the DH5α bacterial competent cells. Positive bacterial colonies were screened through Kanamycin antibiotics LB solid culture medium. Plasmids were extracted and identified by HinIII and EcoRI enzyme digestions, and a result is shown in FIG. 1b. The arrow points out the target fragment. The Aβ42 gene sequence was confirmed by sequence analysis.

Expression of Senile Dementia Aβ42 Plasmid in Eukaryotes.

A BHK cell line was transfected by pVAX-1 Aβ42 by using a liposome method, cells were harvested after 48 hours, total RNAs (TRIZOL, DingGuo Biotech. Co., Ltd) were extracted and reversely transcribed into cDNA in accordance with the RNA RT-PCR operating instruction of TaKaRa Biotechnology (Dalian) Co., Ltd. 1 μg of purified total RNA was placed in a 250 μL centrifuge tube, then related reagents were sequentially added: 4 μL of MgCl₂, 2 μL of 10× buffer, 8.5 μL of DEPC water, 2 μL of dNTP mixture, 0.5 μL of Oligo(dT)₁₂ primer. The reaction conditions were as follows: 42° C. 30 min, 99° C. 5 min, and 5° C. min. PCR reaction was carried out by using the above primer and using a first strand cDNA product as a template, and the expression stripe of Aβ42 was detected. As shown in FIG. 2, in the cells transfected by the pVAX1-Aβ42 plasmid group, a target stripe was amplified through an RT-PCR method. A negative control (−) was an RT-PCR reaction group without reverse transcriptase, and a positive control (+) was PCR control with the pVAX1-Aβ42 plasmid as a template.

### Example 3

### Prokaryotic Expression of Aβ42 Protein

Construction of senile dementia Aβ42 prokaryotic expression plasmid. An Aβ42 gene was amplified through PCR under the guidance of a primer 1:

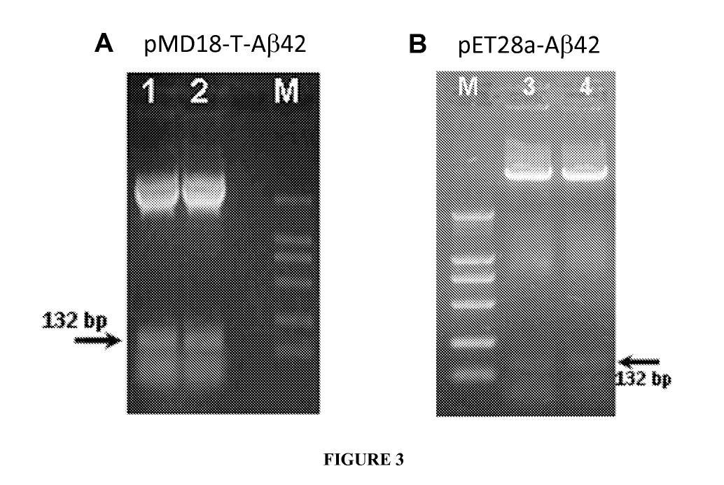

5′-AAAGGATCCATGGATGCAGAATTCC-3′ (SEQ ID NO: 8) and a primer 2: 5′-GCCGTCGACTAACGCTATGACAACA-3′ (SEQ ID NO: 9) (BamHI and Sall recognition sites were respectively introduced to the primers 1 and 2) by using Aβ42-C3d3 as a template. A target fragment was recycled and ligated to a pMD18-T vector. After being identified to be correct through enzyme digestion, the target fragment was sub-cloned to a pET28a vector. The PCR system and a recycling and enzyme digestion identification method were the same as those detailed in Example 1. As shown in FIG. 3, (a) is a result of a pMDI8T-Aβ42 plasmid subjected to BamHI and Sall double enzyme digestion, and (b) is a result of a pET28a-Aβ42 plasmid subjected to BamHI and Sall double enzyme digestion. The results were confirmed with sequence analysis.

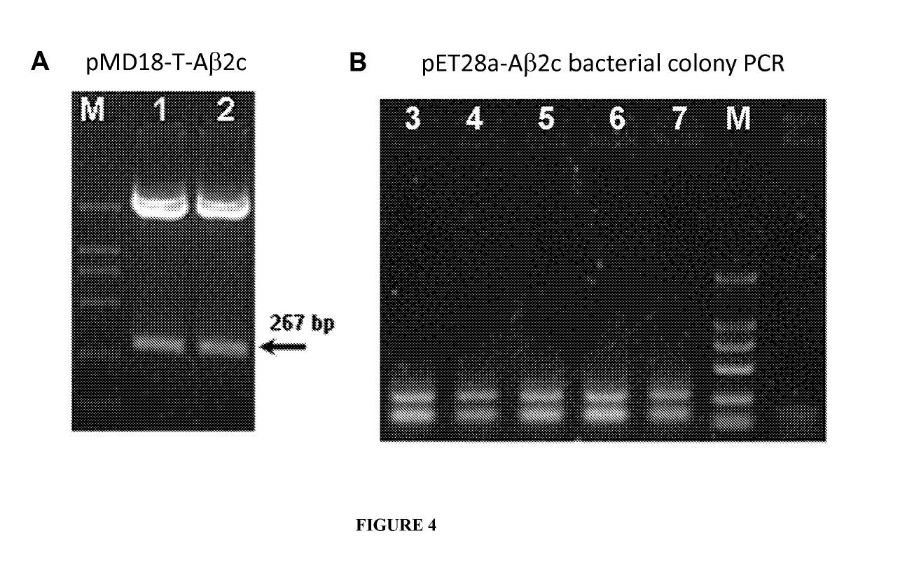

An overlap PCR reaction was carried out under the guidance of a primer 1: 5′-AAAGGATCCATGGATGCAGAATTCC-3′ (SEQ ID NO: 8) and a primer 2: 5′-GCCGTCGACTAACGCTATGACAACA-3′ (SEQ ID NO: 9) (BamHI and Sall recognition sites were respectively introduced to the primers 1 and 2), an overlap DNA fragment 1: 5′-TCGGAA TTCTGCATCTCCTCCTCCCGCTATGACAA-3′ (SEQ ID NO: 10) and a fragment 2: 5′-CGGTGTTGTCATAGCGGGAGGAGGA-3′ (SEQ ID NO: 11) by using pMD18T-Aβ42 as a template to amplify two copies of Aβ42 genes. A reaction system was as follows: 1 μL of plasmid template, 10 pmol of primers 1 and 2, as well as overlap DNA fragments 1 and 2, respectively, 500 mM of KCl, 100 mM of Tris-1-HCl (pH 8.4), 1.5 mM of MgCl₂, 100 μg/mL of BSA, 1 mM of dNTPs, and 2.5 U of Tag DNA polymerase, with the total volume of 25 μL. The reaction conditions were as follows: denaturing for 30 seconds at 94° C., annealing for 35 seconds at 58° C., extending for 30 seconds at 72° C., and conducting total 30 cycles. The two copies of Aβ42 gene fragments were recycled and ligated to a pMD18-T vector. After being identified through enzyme digestion, the target fragment was sub-cloned to a pET28a vector, and positive strains were identified through bacterial colony PCR. FIG. 4 shows pMD 18-T-Aβ422c enzyme digestion and pET28a-Aβ422c bacterial colony PCR electrophoretogram. The enzyme digestion identification method was the same as that of the Example 1.

Prokaryotic Expression of One Copy of Aβ42 Protein and Two Copies of Proteins.

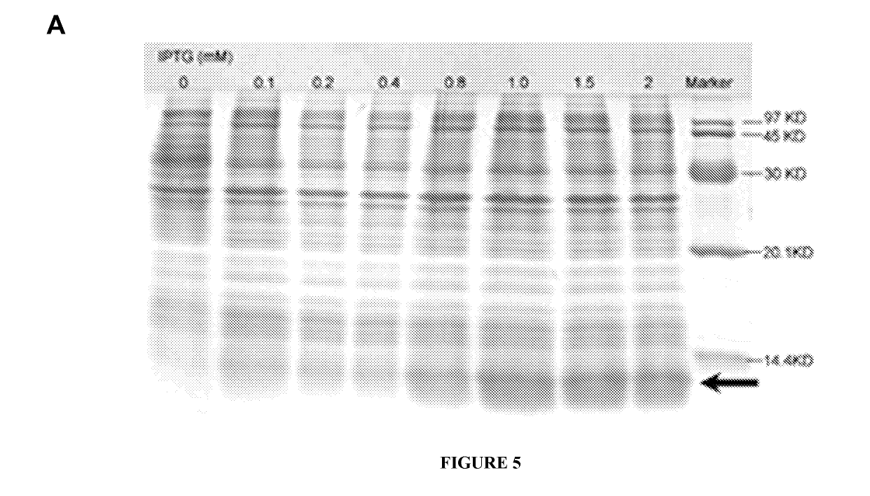

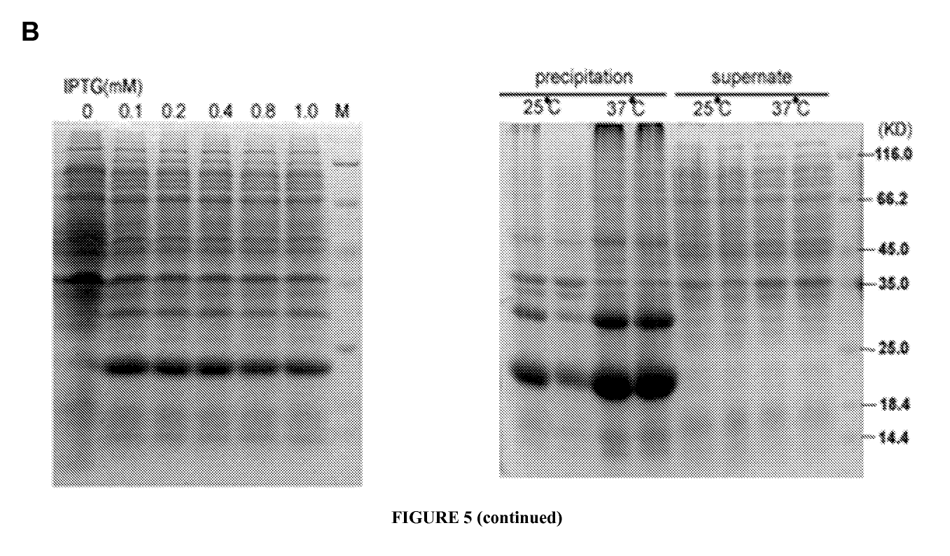

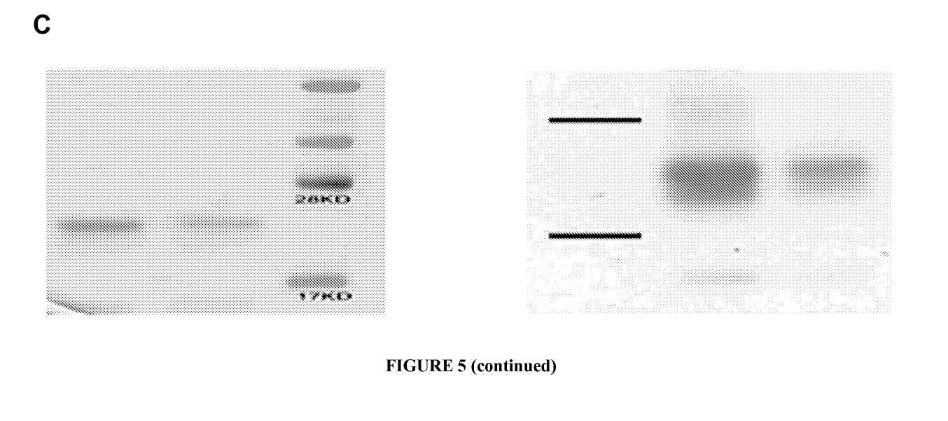

pET28a-Aβ42 and pET28a-Aβ422c plasmids were transformed into BL21 (DE3) prokaryotic expression strain competent cells. After positive strains were screened, the proteins were induced to express by adding IPTG in different concentrations. An induction system comprised 5 mL of bacterial liquid, a temperature of 37° C. or 25° C., and a time of 4 hours. Strains were collected and resuspended in pre-cooling PBS (containing 10 mL/L of Triton X-100 and 1 mg/mL of lysozyme in final concentration), bacteria were lysed by sonication, and the lysed cells were then centrifuged at 12,000 rpm for 15 min at 4° C. The supernatant and precipitant were separated and used for SDS-PAGE detection, wherein the expressed protein was identified as an inclusion body in the precipitate. The size of one copy Aβ42 protein in SDS-PAGE shown in FIG. 5a was about 7 kDa (indicated by the arrow), and the expression quantity was highest when being induced at 1.0 mM IPTG. FIG. 5b (left) shows the expression condition of BL21 positive strains of two copies of Aβ42 recombinant plasmids, induced by proteins of different IPTG concentrations. FIG. 5b (right) shows that the expressed protein was in an inclusion body precipitate form, and the optimal induction temperature was 37° C. The size of the two copies of Aβ42 proteins was 13 KD (indicated by the downward arrow), and a very dense expression stripe (indicated by the upward arrow) was also found corresponding to the double size of the Aβ42 proteins (about 26 kDa). The dipolymer can have been formed because the Aβ42 protein tends to aggregate. In order to confirm that the expressed protein is the Aβ protein, Western Blot was used for detection. The Aβ42 protein was purified through a nickel column (QIAGEN Company), subjected to SDS-page, and transferred to a nitrocellulose membrane. Blocking was carried out in 1% BSA for 1 hour. Primary antibody 6E10 (mouse) anti-Aβ monoclonal antibody (provided by F.L.I. of Leibniz Institute-Fritz Lipmann Institute) was incubated at 1:1000 for 1 hour at room temperature, the anti-mouse-HRP second antibody (Invitrogen) was incubated at 1:2000 for 1 hour at room temperature, and developing was performed. The results are shown in FIG. 5c, wherein the left figure is a SDS-PAGE stained with Coomassie brilliant blue, and the right figure is a Western Blot. Arrows indicate the two copies of Aβ42 proteins and the dipolymer of the two copies of Aβ42 proteins, respectively (indicated by the upward arrow).

### Example 4

### ELISA Detection and T Cell Reaction Detection of Antibody Producing Condition of Different Mouse Species Through Aβ42 Protein Vaccine and DNA Vaccine Combined Immunization

In order to determine whether Aβ42 protein vaccine and DNA vaccine combined immunization can cause immunosuppression without affecting the generation of antibody, two species of mice (Balb/e and C57BL/6, aged 6-8 weeks) were selected for immunization, and the antibody IgG and T-cell proliferation were detected and measured.

ELISA detection of antibody producing condition of Balb/e and C57BL/6 mice through Aβ42 protein vaccine and DNA vaccine combined immunization. Sixteen BALB/c or C57BL/6 female mice aged 6-8 weeks were divided into 4 groups, with four in each group. The first group received intramuscular injection of 50 microliters of PBS solution containing 50 micrograms of pVAX1-Aβ42 plasmid DNA; the second group received subcutaneous immunization of 50 microliters of protein antigen containing 50 micrograms of one copy of Aβ42 protein and ½ volume of completely emulsified Freund's complete adjuvant; the third group received subcutaneous immunization of 50 microliters of protein antigen containing 50 micrograms of one copy of Aβ42 protein and ½ volume of completely emulsified Freund's complete adjuvant and intramuscular injection of 50 microliters of PBS solution containing 50 micrograms of pVAX1-Aβ42 plasmid DNA; and the fourth group was an untreated naive group Immunizations were boosted again by using the same injection mode and dosage at day 14, and the antibody titers of serum at the day 14 and day 28 after the second immunization were measured by using an ELISA method. The detection method comprised the following steps: coating a 96-well ELISA plate with 10 μg/mL of Aβ42 protein antigen; staying overnight at 4° C., blocking in 3% calf serum for 1 hour at 37° C.; washing three times with PBST (0.05% Tween20 was dissolved into PBS), each washing for 5 minutes; adding immunized mice serum of different dilutions; taking unimmunized mice serum as a control; incubating for 1 hour at 37° C.; washing the plate three times by using PBST; then adding 100 μL of horse radish peroxidase-labeled goat anti-mouse IgG (second antibody, Sigma, St. Louis) to each well; incubating for 1 hour at 37° C. and then removing; washing three times with PBST (each wash for 5 min); washing three times with PBST; adding 100 μL of substrate TMB liquid; developing for 20 min at room temperature; stopping the reaction by using 2 M sulfuric acid; and measuring OD_{450/620} optical density values by using a microplate reader. The results of the Balb/e mice are shown in FIG. 6a. The comparison of OD values of IgG of the serum diluted in 400 times and collected at day 14 and day 28 after second immunization indicated that the OD value of the co-immunization group was higher. The OD value of C57 mice are shown in FIG. 6b, and the results were similar with the former.

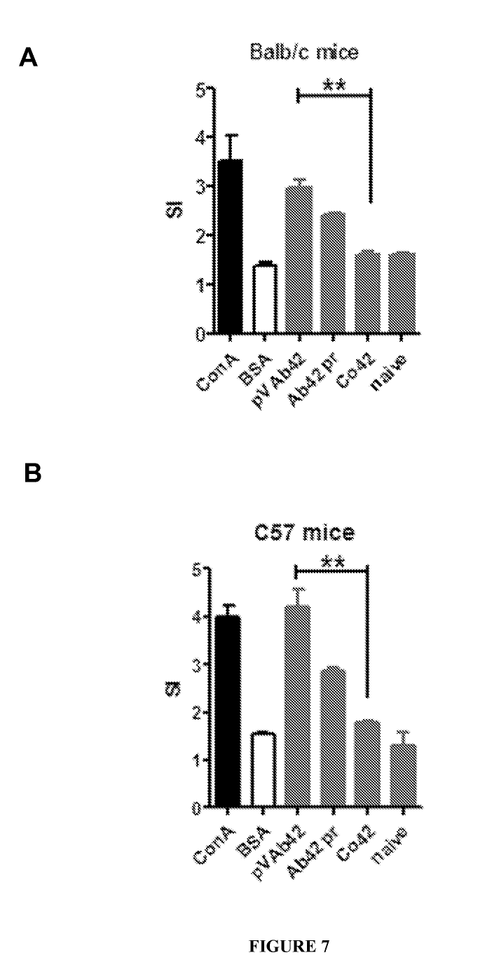

Detection of T-cell reaction condition after Aβ42 protein vaccine and DNA vaccine combined immunization of Balb/c and C57BL/6 mice. The immunized mice of each group were boosted again by using the same method and dosage, and seven days later, a T-cell amplification experiment was carried out by using a MTT method. The method included the following steps: preparing single cell suspension from spleen under an aseptic condition; removing erythrocytes by using an erythrocyte lysis solution; then washing three times with PBS liquid; passing the cell suspension through a sterile glass cotton column to remove B cells; counting cells; adjusting the concentration of the cells to be 3×10⁶ cells/mL; dividing the cell suspension of each group into four parts; adding the cell suspension to a 96-well flat-bottom cell culture plate; and setting three repeating wells for each part, wherein 20 μL of ConA (mitogen) was added to the first part until the final concentration reached 10 μg/mL, a corresponding specific antigen (Aβ42) as a stimulant was added to the second part until the final concentration reached 10 μg/mL, the third part was free of a stimulant, and BSA was added to the fourth part until the final concentration reached 2 μg/mL as a unrelated antigen control; culturing for 72 hours in an incubator at 37° C.; adding 20MTT to each well until the final concentration reached 1 mg/mL; centrifuging the 96-well plate 4 hours later; removing supernatant; adding 150 μL of dimethyl sulfoxide (DMSO) into each well so that the dimethyl sulfoxide was completely dissolved; reading OD values at 570 nm on the microplate reader; and calculating the result according to the following: Stimulation Index SI=(OD value of each stimulated well-OD value of culture medium)/(OD value of unstimulated well-OD value of culture medium). The results are shown in FIG. 7a and FIG. 7b. The T-cell proliferation experiment of two immunized mouse species indicated that the SI stimulation index of the Co42 group was remarkably reduced and greatly differed from that of an independent immunoprotein or DNA group (p<0.03).

### Example 5

### Detection of Effect of Aβ42 Protein Vaccine and DNA Vaccine Co-Immunization on Old C57 Mice

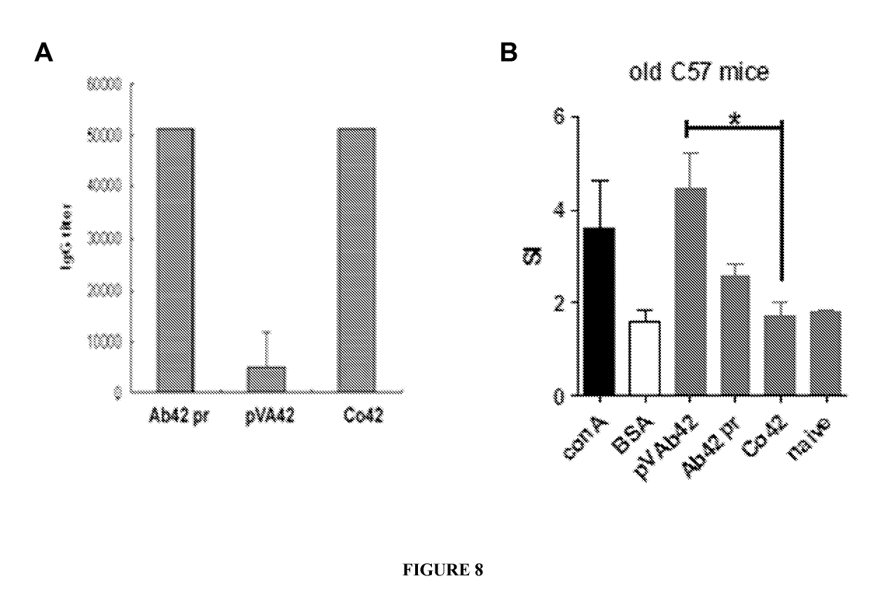

The subsequently selected experimental animals were C57 mice because in the field of senile dementia research, mouse models suffering from senile dementia are commonly selected from C57BL/6. After the animals grow old, the immune systems can degenerate, and so it was determined whether the immune systems of old immunized mice can have the same effect as those of young mice. Old mice of 12 months to 14 months old were selected for performing experiments. 12 old mice of about 12 months old were divided into four groups, with three in each group. The first group received intramuscular injection of 50 microliters of PB solution containing 50 micrograms of pVAX1-Aβ42 plasmid DNA; the second group received intramuscular injection of 50 microliters of protein antigen containing 50 micrograms of one copy Aβ42 protein and ½ volume of completely emulsified Freund's complete adjuvant; the third group received intramuscular injection of 50 microliters of protein antigen containing 50 micrograms of one copy Aβ42 protein and ½ volume of completely emulsified Freund's complete adjuvant and 50 microliters of PBS solution containing 50 micrograms of pVAX1-Aβ42 plasmid DNA simultaneously; and the fourth group was an untreated naive group. Immunizations of the mice were boosted again by using the same injection mode and dosage at day 14. Serum was collected at day 14 after the second immunization. The level of antibody IgG was detected by using an ELISA method, and it was considered as positive when the OD value of an experimental well was twice the OD value of a control well. Immunization was boosted for the last time, and T-cell proliferation detection was carried out by using the MTT method after seven days. The results are shown in FIG. 8, wherein (a) is a comparison of titers of Aβ42 antibody and IgG, demonstrating that the titers of the co-immunization Co42 group and the separate immunoprotein group both reached very high levels (at least 51,200 times), and wherein (b) are the results of T-cell proliferation, indicating that the T-cell reaction was inhibited with significant difference compared to that of the separate immunization group (p<0.05).

### Example 6

### Dosage Detection of Aβ42 Protein Vaccine and DNA Vaccine Co-Immunization

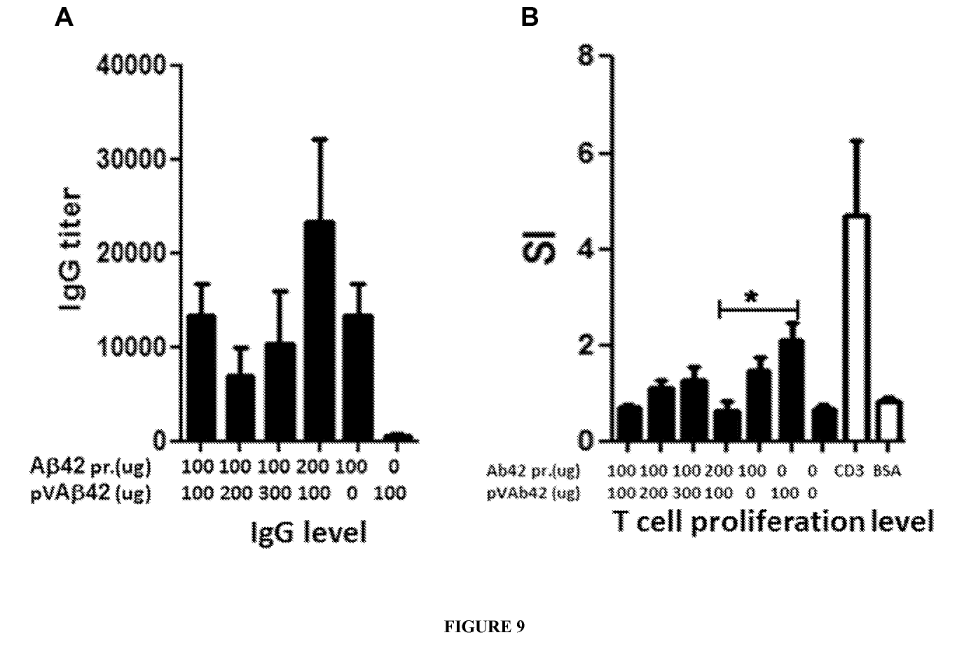

In order to determine an appropriate dosage of Aβ42 protein vaccine and DNA vaccine co-immunization, C57 mice were immunized by using different dosage combinations. Twenty-one C57 female mice in 8-week old were divided into seven groups, with three in each group. The first group received intramuscular injection of 100 microliters of PBS solution containing 100 micrograms of pVAX1-Aβ42 plasmid DNA; the second group received intramuscular injection of 100 micro liters of PBS solution containing 100 micrograms of two copies of Aβ42 protein; the third group received intramuscular injection of 200 micro liters of PBS solution containing 100 micrograms of pVAX1-Aβ42 plasmid DNA and 100 microliters of PBS solution containing 100 micrograms of two copies of Aβ42 proteins simultaneously; the fourth group received intramuscular injection of 100 microliters of PBS solution containing 100 micrograms of pVAX1-Aβ42 plasmid DNA and 100 microliters of PBS solution containing 100 micrograms of two copies Aβ42 proteins simultaneously; the fifth group received intramuscular injection of 100 microliters of PBS solution containing 100 micrograms of pVAX1-Aβ42 plasmid DNA and 100 microliters of PBS solution containing 200 micrograms of two copies of Aβ42 proteins simultaneously; the sixth group received intramuscular injection of 100 microliters of PBS solution containing 300 micrograms of pVAX1-Aβ42 plasmid DNA and 100 microliters of PBS solution containing 100 micrograms of two copies Aβ42 proteins simultaneously; and the seventh group was an untreated naive group. Immunizations of the mice were boosted again by using the same injection mode and dosage at day 14. Serum was collected at day 14 after the second immunization. The level of the antibody IgG was detected using an ELISA method. Immunization was boosted for the last time, and 7 days later, a T-cell proliferation detection experiment was conducted by using an MTT method. As shown in FIG. 9, when the dosage of DNA immunization was 100 μg, the anti-Aβ42 antibody concentration increased as the quantity of mixed proteins increased. However, T-cell proliferation was not at the linear increase tendency. Immunosuppression occurred with 100 μg DNA and 100 μg Aβ42 protein as well as when 100 μg DNA and 200 μg Aβ42 protein were mixed. The administration of 100 μg DNA and 200 μg Aβ42 protein mixture was significantly different (p<0.05).

### Example 7

### Long-Term Effect Experiments of Aβ42 Antigen Combined Immunization

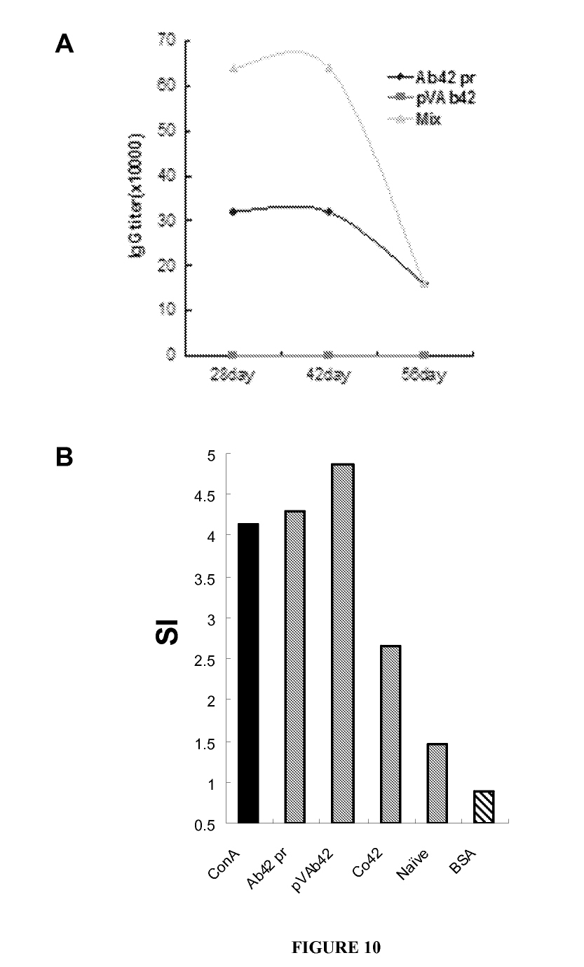

In order to detect the duration of immunization effects after the mice are immunized, the long-term effect of Aβ42 antigen protein and DNA combined immunization effects was evaluated. Sixteen BALB/c or C57BL/6 female mice aged 6-8 weeks were divided into four groups, with four in each group. The first group received intramuscular injection of 100 microliters of PBS solution containing 100 micrograms of pVAX1-Aβ42 plasmid DNA; the second group received intramuscular injection of 100 microliters of PBS solution containing 100 micrograms of two copies Aβ42 proteins; the third group received intramuscular injection of 100 microliters of PBS solution containing 100 micrograms of pVAX1-Aβ42 plasmid DNA and 100 microliters of PBS solution containing 100 micrograms of two copies of Aβ42 proteins simultaneously; and the fourth group was an untreated naive group. Immunizations of the mice were boosted twice by using the same injection mode and dosage at day 14 and at day 28; serum of the mice was collected at day 28, day 42, and day 56 after the third immunization, and the level of the antibody IgG was detected by using an ELISA method. The mice were sacrificed after the serum was collected at the last time, and a T-cell proliferation experiment was conducted by using an MTT method. As shown in FIG. 10a, the antibody titer of the co-immunization group declined from the last immunization to the day 56, but was still at a higher level (128,000). The antibody titer of the DNA immunization group was always low, which was related to TH1 reaction mechanism induced by DNA immunization, and to the immunization dosage and no immunologic adjuvant. The results of T-cell proliferation is shown in FIG. 10b, indicating that the immunosuppression of combined immunization still existed, and T-cells were not activated for a long period of time after co-immunization. This indicated that the immunization strategy can be used as a long-term therapy means.

### Example 8

### Detection of Binding Capacity of Antiserum and Aβ Fiber

An Aβ protein vaccine functions in the removal of deposited intracerebral Aβ therapeutic protein via the Aβ antibody generated in an organism and alleviates clinical symptoms of patients with senile dementia. It was determined whether or not the generated antibodies effectively bind to Aβ fiber protein to confirm their functional effectiveness. The following methods were used for indirect verification.

Dot Blot Detection of Binding of Antiserum and Aβ Protein Fiber.

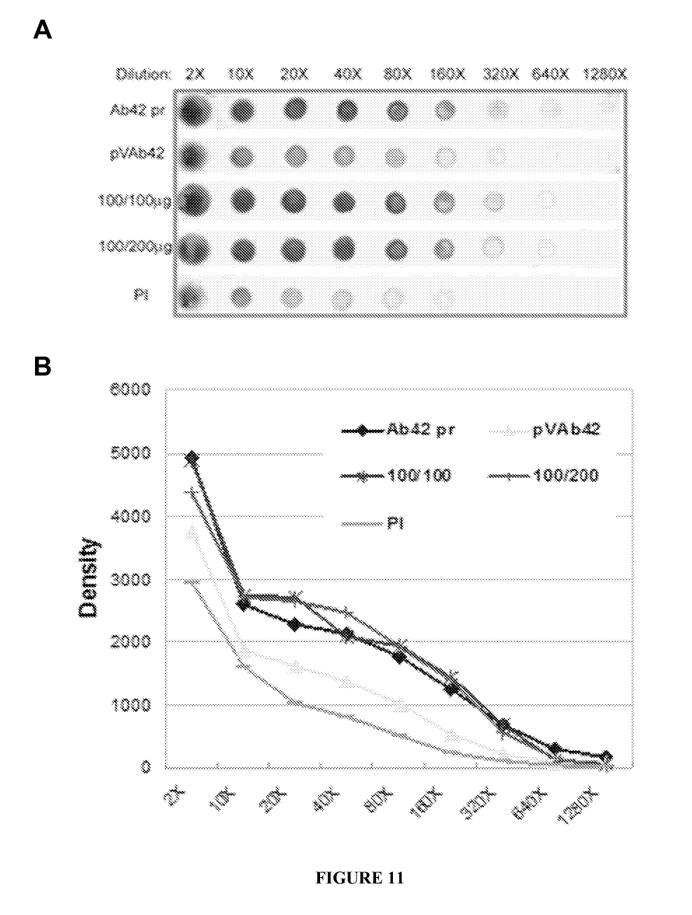

The serum collected from different immunization groups (the serum of the mice of the same group is mixed together) were diluted according to certain titer and dotted to a nitrocellulose membrane (3 μL for each titer). After liquid was dried completely, the membrane was put into TBS solution containing 2% BSA, and the membrane was shaken at room temperature and blocked for 40 min. The membrane was washed twice with double distilled water, and then washed once with 1×TBST. The membrane was put into an Aβ40 protein TBS diluent (to a final concentration of 1 μg/mL) and shaken and incubated for 1 hour at room temperature. The membrane was washed three times with 1×TBST, each wash for 5 min. The membrane was put into a 2D8 anti-Aβ antibody TBS diluent (1:1000) and shaken and incubated for 1 hour at room temperature. The membrane was washed three times with 1×TBST, each wash for 5 min. The membrane was put into an α-His antibody-HRP TBS diluent (1:2000) and shaken and incubated for 1 hour at room temperature. The membrane was washed three times with 1×TBST, each wash for 5 min. An ECL was used for developing. Results are shown as FIG. 11a, indicating that the amount of bound Aβ fibers (provided by F.L.I.) in the 200 μg protein and 100 μg DNA co-immunization group was the most with the strongest binding capacity, the amount of bound Aβ fibers in the 100 μg protein and 100 μg DNA co-immunization group was second to that of the 200 μg protein and 100 μg DNA co-immunization group, the amount of bound Aβ fibers in the single 100 μg protein group followed that of the 100 μg protein and 100 μg DNA co-immunization group, and the amount of bound Aβ fibers in the single 100 μg DNA group was the lowest. FIG. 11b is a scoring diagram for analyzing density of each point in the Dot Blot. The Dot Blot result is consistent with the result of the antibody IgG titer. The results indicated that the antibody generated by Aβ42 antigen immunization can effectively bind to Aβ protein fiber.

Binding of Antiserum and Aβ Deposition in the Brain of the Transgenic Ill Mouse Suffering from APP/PS1 Senile Dementia.

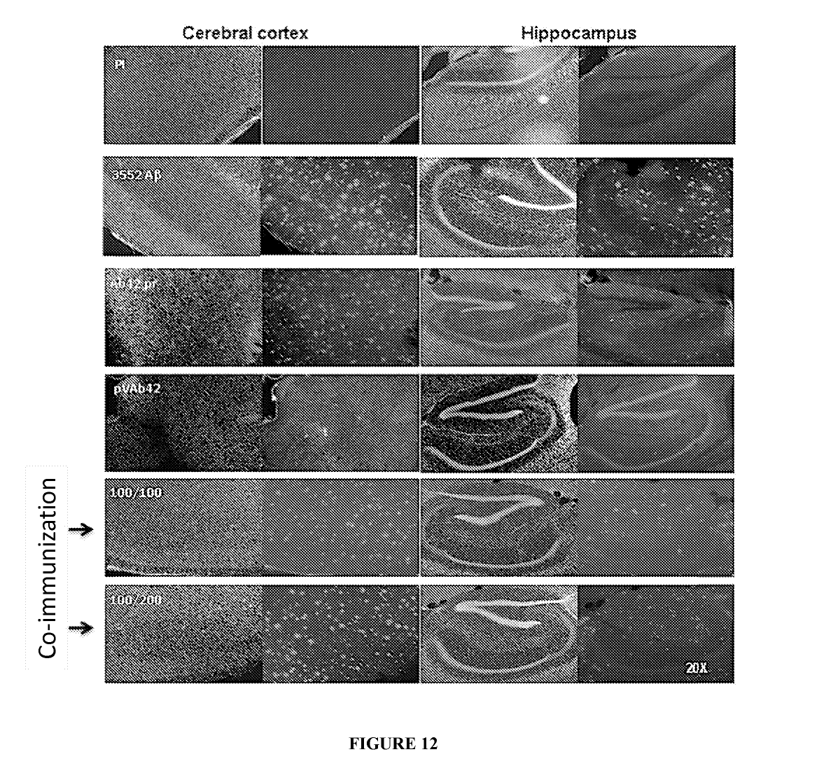

0.4 μm of brain tissue slices of transgenic ill mice suffering from APP/PS1 senile dementia were respectively placed in each well of a 24-well plate, each well containing PBS. Each well was washed twice to three times. The wells were blocked with 10% NGS and 0.2% Triton X100 in PBS for 1 hour at room temperature. The supernatant was removed, and the remaining mixture was washed three times with PBS. The serum derived from different immunization groups and was diluted 1:200. The mixture was incubated overnight at 4° C. The supernatant was removed. The remaining mixture was washed three times with PBS. Gat anti-mouse second antibody (label 488 nm) second antibody diluents (1:1000) was added, and it was incubated away from light at room temperature for 1 hour. The supernatant was removed, the remaining mixture was washed three times with PBS, and the PBS was removed. Dapi (1 μg/mL) was used for counterstaining. 2 drops of Dapi solution were dripped to each tissue, the tissue was placed away from light for 2 min and quickly washed twice with PBS, the tissue was moved to glass slides, and coverslips were placed and sealed. An electron microscope was used for detection. In FIG. 12, blue is a Dapi counterstaining result and displays cells, wherein red is the binding condition of the antibody and Aβ protein in brain. PI was a negative serum group, as the antibody binding to Aβ protein was absent in the serum, and no Aβ spot was stained. 3552 anti-Aβ monoclonal antibody (provided by F.L.I.) was used as a positive control, and a large number of Aβ spots were observed in both the hippocampus and the cerebral cortex in the brain tissues of ill mice. In the experiment groups, many focus spots were stained in the serum of the immunization group containing Aβ42 protein, which indicated that antibody against Aβ in the serum was capable of binding to the Aβ protein in the brain tissues. Aβ spots were hardly seen to be stained in the single immunization DNA group due to very low antibody titer.

### Example 9

### Detection of Cytokine Level

Twelve BALB/c or C57BL/6 female mice (aged 6-8 weeks) were divided into four groups, with three in each group. The first group received intramuscular injection of 100 micro liters of PBS solution containing 100 micrograms of pVAX1-Aβ42 plasmid DNA; the second group received intramuscular injection of 100 microliters of PBS solution containing 100 micrograms of two copies of Aβ42 proteins; the third group received intramuscular injection of 100 micro liters of PBS solution containing 100 micrograms of pVAX1-Aβ42 plasmid DNA and 100 microliters of PBS solution containing 100 micrograms of two copies Aβ42 proteins simultaneously; and the fourth group was an untreated naive group. Immunization of the mice was boosted once by using the same method and dosage at day 14; RT-PCR detection and flow-cytometry detection of the cytokines were carried out at day 7 after immunization. An RT-PCR detection method comprised the following steps: decapitating the immunized mice, taking out a spleen, extracting total RNA (TRIZOL, DingGuo Biotech. Co., Ltd), reversely transcribing RNA into cDNA in accordance with the RNA RT-PCR operating instruction of TaKaRa Biotechnology (Dalian) Co., Ltd., placing 1 μg of purified total RNA in a 250 μL centrifuge tube, and then sequentially adding related reagents: 4 μL of MgCl₂, 0.2 μL of 10× buffer, 8.5 μL of DEPC water, 2 μL of dNTP mixture, 0.5 μL of RNase inhibitor, 0.5 μL of 1M-MLV reverse transcriptase (Promaga Company), and 0.5 μL of Oligo(dT)₁₂ primer. The reaction conditions were as follows: 42° C. for 30 min, 99° C. for 5 min, and 5° C. for 5 min. PCR amplification was carried out by using a house-keeping gene of hypoxanthine phospho-ribosyl transferase (HPRT) as an endogenous expression standard, the cDNA concentrations of each group were adjusted to be the same. PCR amplification of other cytokines was carried out, wherein primers and PCR reaction conditions required for the reaction are shown in Table 1.

TABLE 1
Primer sequences of HPRT, TGFβ, and IL-10 and PCR reaction parameters.

| TARGET | | REACTION |
|---|---|---|
| Gene | PRIMERS | CONDITIONS |
| HPRT | 5′GTTGGATACAGGCCAGACTTTGTTG (SEQ ID | 94° C. sec, 60° C. |
| | NO: 12) | 30 sec, and 72 C.40 |
| | 3′GAGGGTAGGCTGGCCTATGGCT (SEQ ID NO: | sec |
| | 13) | |
| TGFβ | 5′-CTCCCACTCCCGTGGCTTCTAG(SEQ ID NO: | 94° C. 30, 55° C. 30 |
| | 14) | sec, and 72° C. 40 sec |
| | 3′-GTTCCACATGTTGCTCCACACTTG (SEQ ID | |
| | NO: 15) | |
| IL-10 | 5′CCAGTTTTACCTGGTAGAAGTGATG (SEQ ID | 94° C. 30, 56° C. 30 |
| | NO: 16) | sec, and 72° C. 40 sec |
| | 3′TGTCTAGGTCCTGGAGTCCAGCAGACTCAA | |
| | (SEQ ID NO: 17) | |

The method for detecting the cytokines by using an intracellular cytokine staining method comprised the following steps: placing T-cells sterilely separated from the spleens of the immunized mice in a 10% culture medium, diluting into 1×10⁷ cells/mL, adding 100 μL to a 96-well cell plate, simultaneously adding an antigen with the final concentration of 10 μg/mL, optionally adding a co-stimulating signal of CD28 monoclonal antibody with the final concentration of 10 μg/mL, uniformly mixing, culturing in 5% CO₂ at 37° C., stimulating for 4-6 hours, and then adding 1 μL of monensin protein transport inhibitor into each well, treating with monensin for 2 hours, then centrifuging for 5 min with 2 mL of PBS at 2,000 rpm, and resuspending the cells in 50 μL of PBS, adding 1.0 μL (0.5 mg/mL) of Fe receptor antibody, blocking the Fe receptor, keeping in ice bath for 20 min, adding 2-4 mL of PBS, centrifuging for 5 min at 2,000 rpm, and removing supernatant to obtain 50 μL of PBS suspension cells, resuspending the cells in 200 μL of the PBS in which 4% paraformaldehyde was added, incubating for 10-15 min at room temperature and centrifuging for 5 min at 2,000 rpm by using 2-4 mL of PBS, resuspending the cells in 200 μL of 0.1% saponin, incubating for 7 min at 4° C., centrifuging for 5 min at 2,000 rpm by using 2-4 mL of PBS to obtain 50 μL of PBS suspension cells, adding a proper amount of direct-labelled cytokine fluorescence antibodies and surface molecule antibodies, keeping an ice bath for 30 min, adding 2-4 mL of PBS, centrifuging for 5 min at 2,000 rpm, removing the supernatant, and treating before loading by taking 300-400 μL of PBS suspension cells, filtering the cell suspension into an FACS special tube by using a 200-mesh copper wire mesh, and detecting and analyzing using equipment.

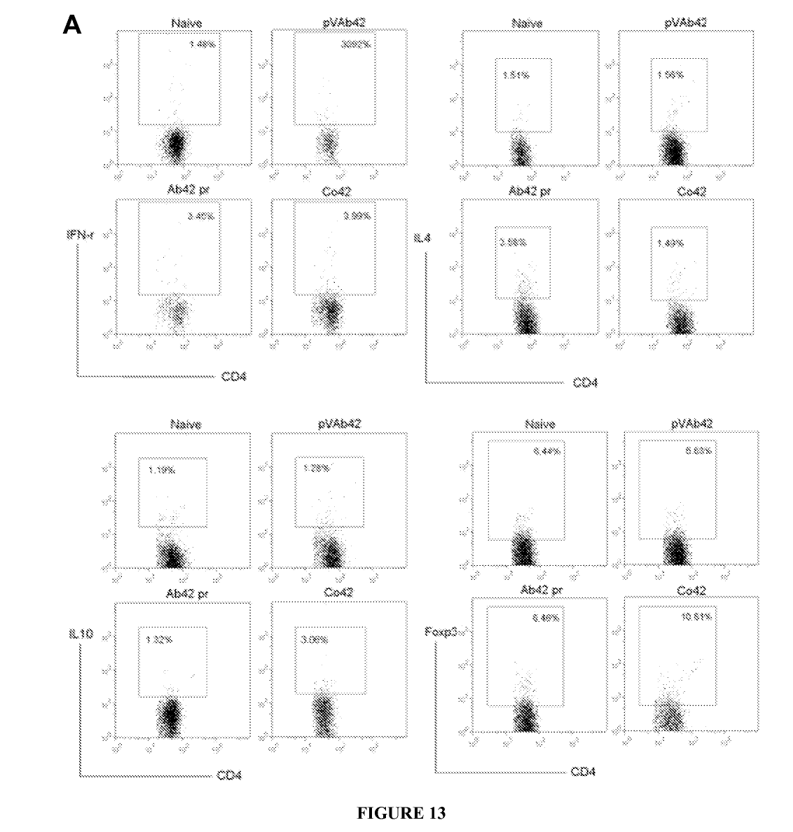

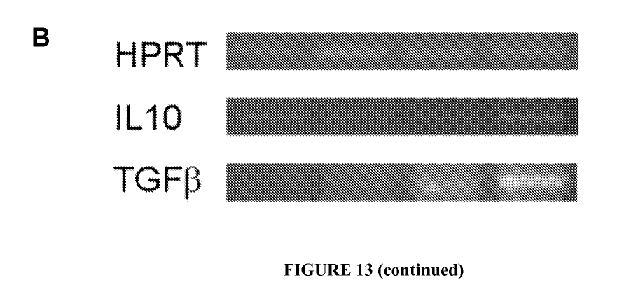

The results are shown as in FIG. 13a, wherein the change of CD4-IFNγ in the immunization group was not obvious, however, the CD4-IL4 expression amount in the Aβ42 protein immunization group was greater. IL4 was closely associated with inflammatory response. When co-immunized with Aβ42 protein and DNA, the expression amount of IL4 declined, indicating that co-immunization can inhibit the expression of IL4 and be related to inhibition of the inflammatory response. In addition, CD4-IL10 and foxp3 were highly expressed in the protein and DNA co-immunization group, and the occurrence of immunosuppression can be related to the regulatory T-cells that induce the highly expressed foxp3 and IL10. In addition, the RT-PCR result in FIG. 13b shows that T cells highly expressed IL10 and TGFβ.

### Example 10

### Immunohistochemical Staining Condition of CD4+ T Cells in Brain Tissues of Immunized Mice

The brain tissues of the mice were from different immunization groups of old mice as in Example 8 above. The tissue slices were stained by using an immunohistochemical SABC method. Briefly, tissues were embedded by first adding liquid paraffin to an iron die cooling slightly, placing the brain tissues fixed by using 4% formaldehyde into paraffin, arranging orderly, covering a plastic die box, and finally adding a small amount of liquid paraffin. The tissue samples were refrigerated so that the paraffin turned into a solid state. For slicing, the embedded tissues were taken from the die and placed on a paraffin slicing machine. The tissues and the cutting direction were the same by regulating the upward, downward, leftward, and rightward directions of the tissues. The slicing thickness (5 μm) was regulated. A sliced glass slide was pulled outwards using a writing brush. The glass slide containing a complete tissue was placed into warm water at 40° C. using a pincet. Air bubbles were dispersed in the water bath before the tissue glass slide was placed into the warm water at 40° C. The tissues were expanded due to heating. The tissue samples were fished out using the glass slide and placed into an incubator at 37° C. To deparaffinate the samples, the glass slide was sequentially placed into dimethylbenzene, dimethylbenzene, 100% alcohol, 100% alcohol, 95% alcohol, 90% alcohol, 80% alcohol, and 70% alcohol, wherein the tissue was kept in each reagent for 10 min.

Antigen repair was performed using an autoclave. Briefly, the tissue samples were washed for a certain period of time in clear water after deparaffinating. 3% H₂O₂ was added to soak for 10 min to remove endogenous catalase. H₂O₂ was poured away and the samples were washed twice in clear water. Citrate buffer solution was added and the samples were placed in the autoclave at 120° C. for 10 min so as to expose sites of an antigen.

The samples were cooled to room temperature. The citrate buffer solution was poured away. The samples were washed twice with water. The glass slide was placed in the PBS for 5 min and washed twice. The PBS solution was dried around the tissues. Serum was added immediately to ensure that some nonspecific sites were blocked. The samples were then placed in the incubator at 37° C. for 30 min. The serum was diluted by 10 times (900 μL of PBS: 100 μL of serum blocking liquid). The glass slide was taken out of the incubator. The serum around the tissues was dried on the front and back surfaces of the glass slide by using absorbent paper. The primary antibody of CD4 antibody (from Rat) was added, and the slides were stored overnight in a refrigerator of 4° C. The glass slide was taken from the refrigerator, placed in PBS, and washed three times, with each wash 5 min. The PBS around the tissues was dried. A secondary antibody (HRP-anti-Rat antibody) was added, and the slides were placed in an incubator of 37° C. for 30 min. The slice was taken from the incubator, placed in PBS, and washed three times, with each wash 5 min. The PBS around the tissues was dried. SABC was added and the slides were placed in the incubator of 37° C. for 30 min. The SABC was diluted by 100 times (990 μL PES: 10 μL SABC).

The slice was taken from the incubator, placed in PBS, and washed three times, each wash 5 min. The PBS around the tissue was dried and color developing agent was added. The color developing agent was prepared as follows: 1 drop of color developing agent A was added into 1 mL of water with uniform shaking. 1 drop of color developing agent B was added with uniform shaking. 1 drop of color developing agent C was added with uniform shaking. Color developing agent A was DAB, color developing agent B was H₂O₂, and color developing agent C was phosphate buffer. The slice was placed into water to be washed. The glass slide was sequentially placed into 70% alcohol, 80% alcohol, 90% alcohol, 95% alcohol, 100% alcohol, 100% alcohol, dimethylbenzene and dimethylbenzene, wherein the slice stayed for 2 min in each reagent, respectively. The slice was finally soaked in dimethylbenzene and moved to a ventilation cabin. To mount, neutral gum was dripped around the tissue and a coverslip was used to cover the tissue.

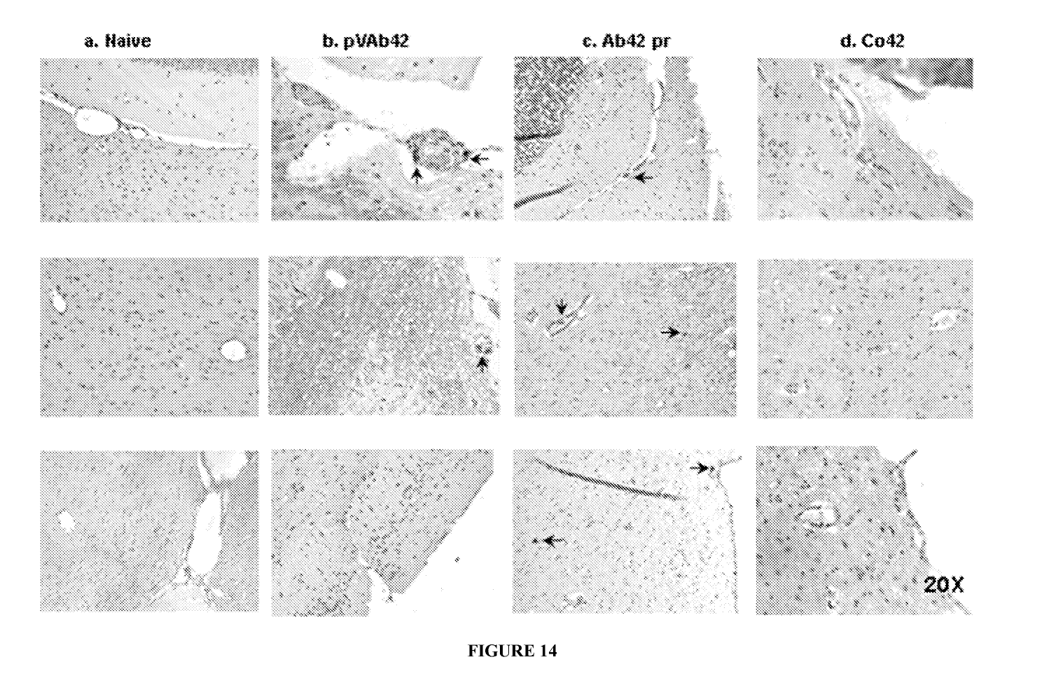

Examination of the Aβ protein senile dementia vaccine was temporarily delayed in phase II clinical trials because meningitis side effects occurred in 5% of patients. Further studies found that the meningoencephalitis was caused by invasion of T lymphocytes in brains and caused inflammation. Other researchers found by using an immunohistochemical staining method that animals immunized by immunoprotein vaccines had lymphocytes in the brain. Therefore, the condition of CD4+ T cells in the brains of immunized mice was determined to examine whether the senile dementia vaccine immunization strategy inhibited the meningitis side effects. As shown in FIG. 14, although a lot of CD4 cellular infiltration was not found in tissue slices, a small number of positive cells (pointed by the arrow) were found near blood vessels or in the blood vessels of brain slices of a separate immunization protein group. Some positive cells were also displayed in the separate immunization DNA group slices, which can be related to stronger Th1 type T cell reaction produced by the DNA vaccine. Abnormal phenomenon in brain blood vessels or around the blood vessels was not seen in the naïve negative control group, and positive T cells were not seen in the Aβ protein and DNA-co-immunization group. Thus the Aβ protein and DNA combined immunization method effectively prevented meningitis.

### Example 11

### Results of Aβ42 Protein and DNA Co-Immunization of APP Senile Dementia Model Mice

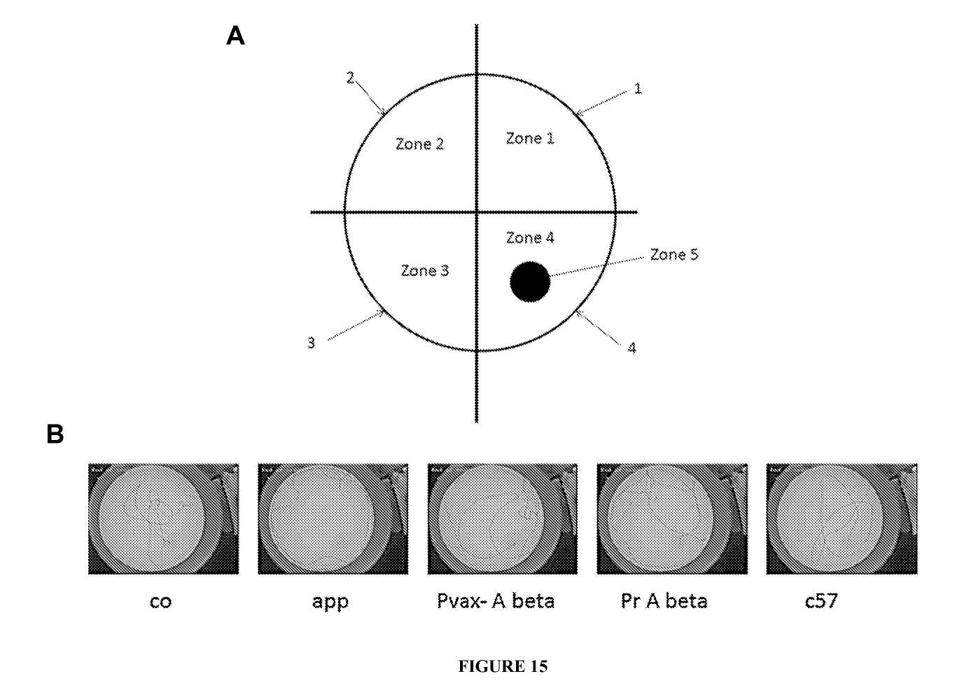

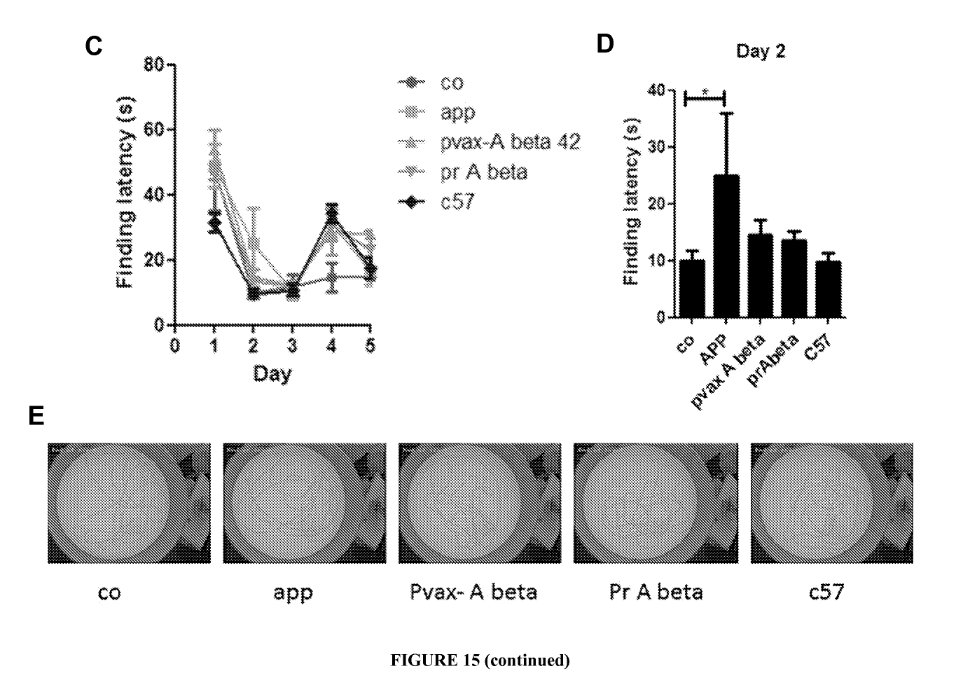

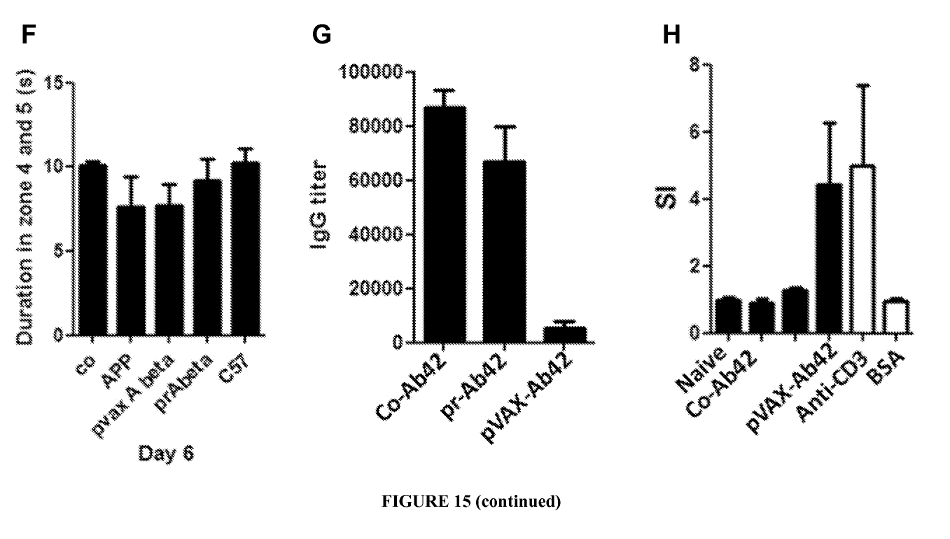

Male APP transgenic mice (aged 11-months) were divided into four groups, with three in each group. The first group received intramuscular injection of 100 microliters of PBS solution containing 100 micrograms of pVAX1-Aβ42 plasmid DNA; the second group received intramuscular injection of 100 microliters of PBS solution containing 100 micrograms of two copies of Aβ42 proteins; the third group received intramuscular injection of 100 microliters of PBS solution containing 100 micrograms of pVAX1-Aβ42 plasmid DNA and 100 microliters of PBS solution containing 200 micrograms of two copies of Aβ42 proteins simultaneously; and the fourth group was an untreated naive group. Immunizations of the mice were boosted using the same method and dosage at day 14 and day 28. Water maze experiments are conducted at day 30 to day 35. FIGS. 15b-15f. Antibodies were detected using the Elisa method (FIG. 15g), and T cell proliferation was detected using the MTT method at day 36 (FIG. 15h).

The water maze experiment method was performed by artificially dividing a water maze into four zones and defining a platform as a fifth zone. FIG. 15a. Each mouse was allowed to dive into the water opposite of the water maze from each zone center point 1, 2, 3, and 4. The experiment was stopped if the mice found the platform within 1 min. The experiment was automatically stopped if the mice did not find the platform at 1 min. The average time of the four times was calculated as the seek time of that day. At day 6, the platform was taken out and the mice were allowed to swim once for 1 min. The average residence time in the fourth zone was calculated.

### Example 12

Aβ42 Co-Immunization Induces High IgG Titer Anti-Aβ42 Antibodies Capable of Binding to Amyloid Plaques

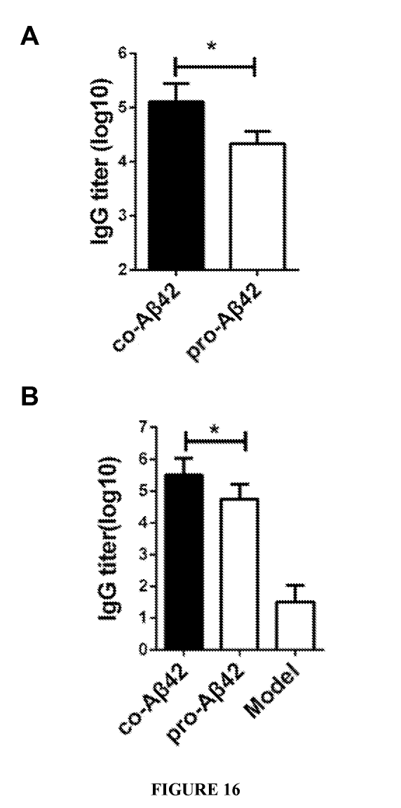

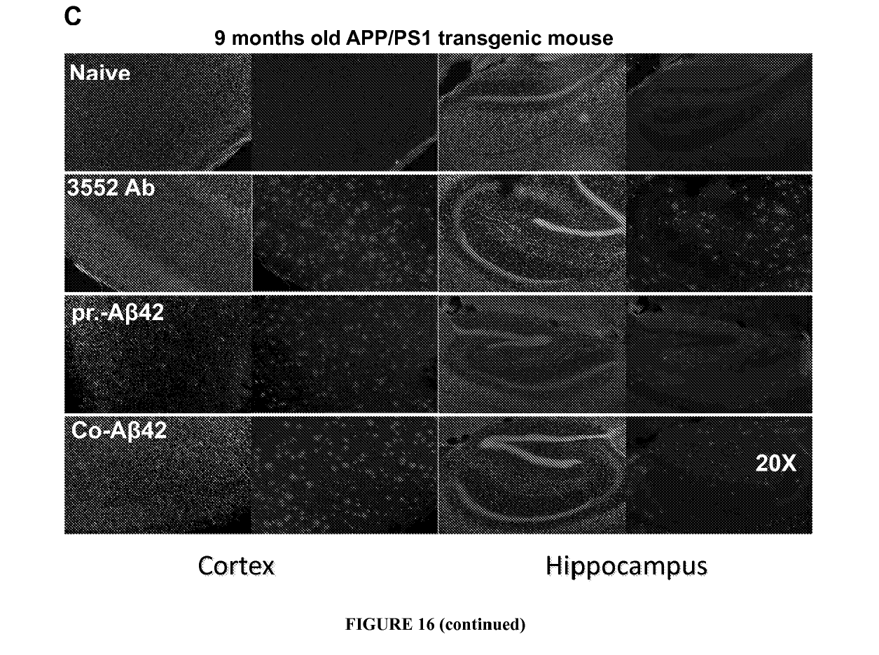

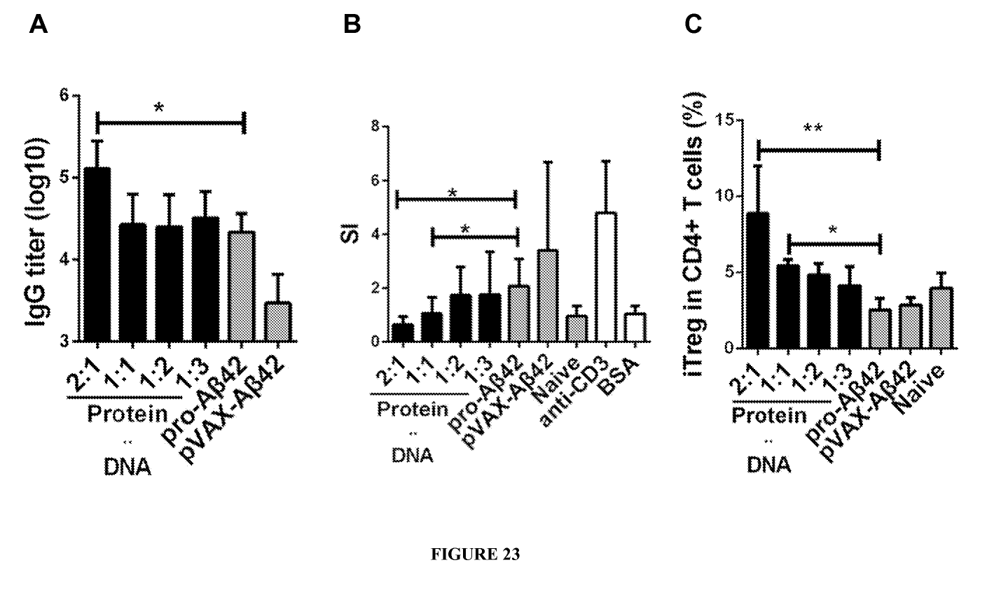

Anti-Aβ antibodies are considered to be the main contributors to the efficacy of AD vaccines. To test if Aβ42 protein vaccine or Aβ42 protein plus Aβ42-coding DNA-vaccine (co-immunization, according to the regimen described in Material and Methods) could elicit a robust anti-Aβ42 immune response, C57BL/6 mice were immunized intramuscularly in biweekly intervals. Serum samples were taken and ELISA was done to measure IgG titers. The co-immunized group had higher IgG titer than the protein-only vaccine group (p<0.05; FIG. 16A), and the IgG titer induced by the DNA vaccine was 2 log₁₀ lower than in the co-immunized group and 1 log₁₀ lower than in the protein vaccine group (FIG. 23A). To further test the specificity of the antibodies generated by immunized mice, sera were used to immunostain amyloid plaques from APPPS1 mice. The APPPS1 mouse is a model of AD characterized by early and massive accumulation of amyloid plaques. As shown in FIG. 16B, serum samples from the co-immunized group strongly reacted with plaques in cortex and hippocampus, similar to the positive control polyclonal anti-Aβ3552. Another well-established model, the APP695 transgenic mouse, was also used. This mouse line harbors an APP695 transgene, and after 8 months the mouse develops amyloid plaques and displays behavioral deficits in the open field test and Morris water maze. To assess whether the co-immunization could also induce anti-Aβ42 IgG in these mice, ELISA was used to measure the IgG titer after the 4th immunization. The result showed that anti-sera from these co-immunized mice also had higher IgG titers than those from protein immunized mice (FIG. 16C). These results demonstrated that the co-immunization strategy resulted in robust production of potentially plaque-clearing anti-Aβ42 antibodies.

### Example 13

### Co-Immunization of Aβ42 Reduces Plaque Formation in APP695 Mice

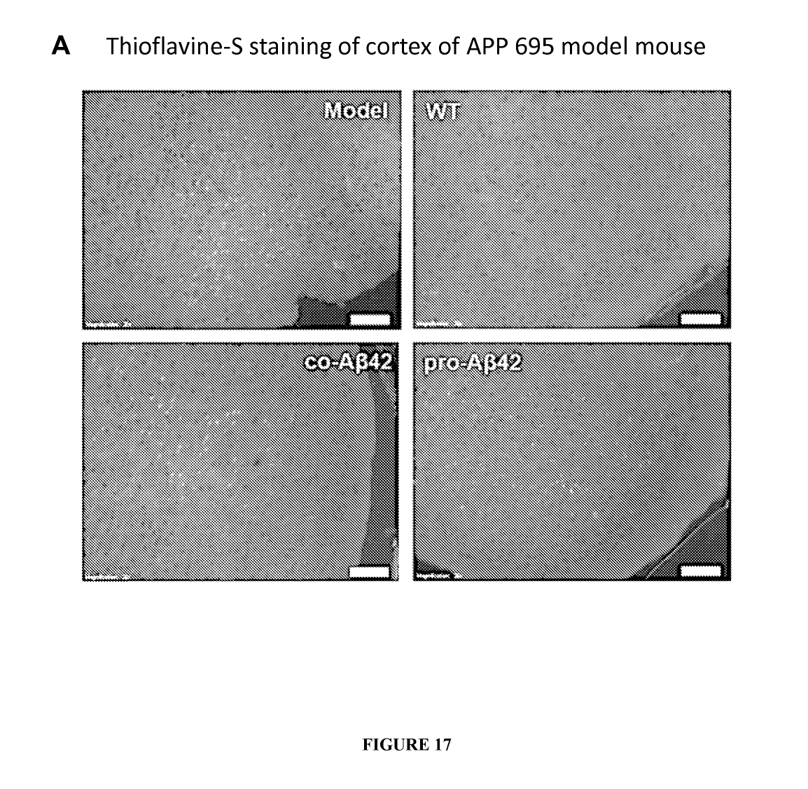

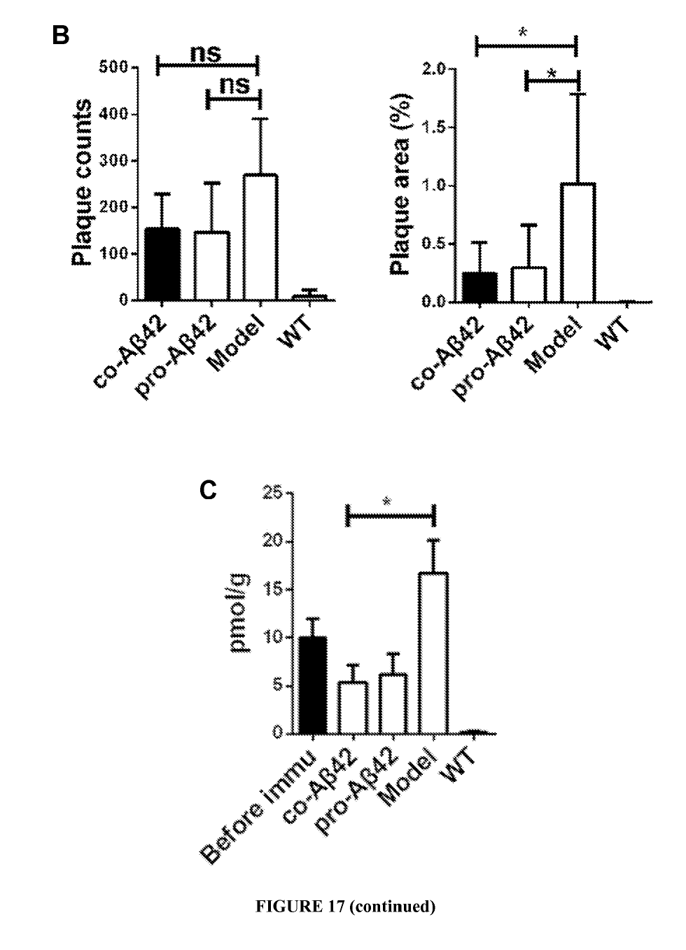

To investigate whether the co-immunization regimen could reduce amyloid plaque formation effectively in vivo, APP695 mice were sacrificed 14 days after the last immunization and analyzed for plaque deposition in their brains using either ELISA against Aβ42 antigen or fibrillar amyloid specific thioflavine-S staining. The ELISA result showed that insoluble Aβ42 depositions in brains were significantly reduced in both co-immunized and protein immunized groups compared to the untreated APP695 mice (p<0.05; FIG. 17C).

Brain sections of cortices were stained with thioflavine-S and the number of plaques as well as total plaque area were determined (FIG. 17A, FIG. 17B). As expected, the APP695 model group had the highest plaque counts, whereas the immunized groups had lower plaque counts, albeit not significantly so. However, the plaque areas were significantly smaller in both the co-immunization group and protein immunized groups compared to the APP695 model group (FIG. 17B). In summary, these results indicated that both the co-immunization and protein regimens effectively reduced plaque loads in APP695 mice.

### Example 14

### Co-Immunization of Aβ42 Attenuates the Abnormal Exploratory Activity of APP695 Mice in the Open-Field Test

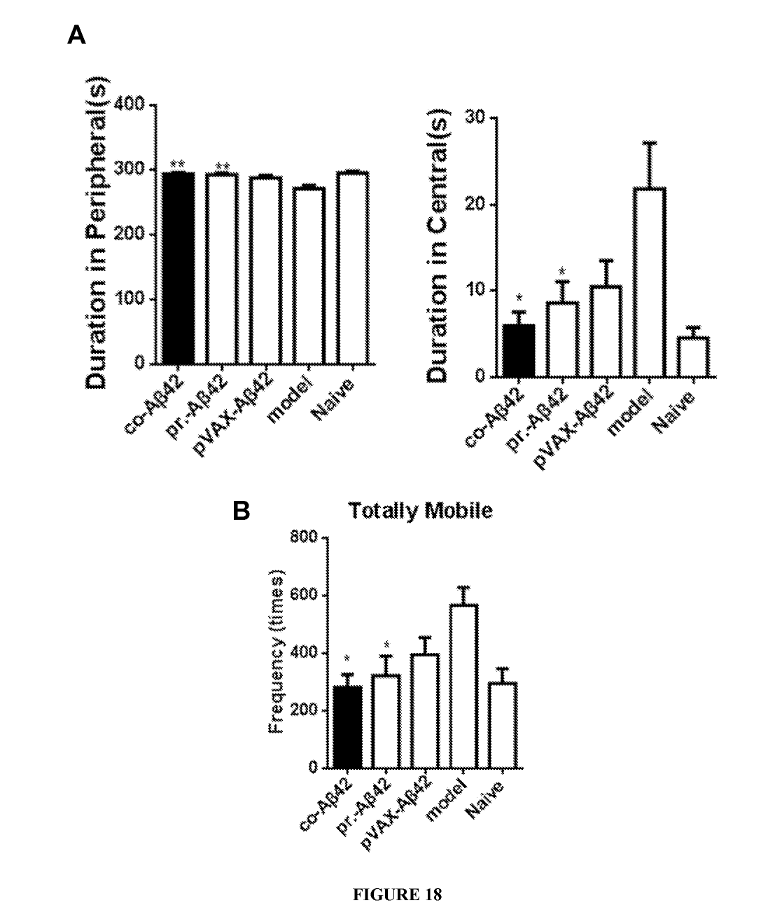

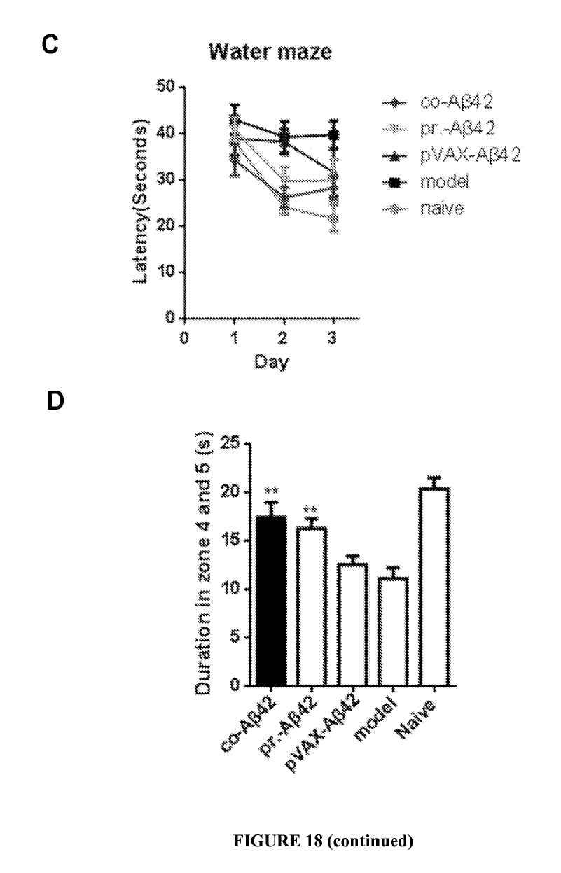

The open field test (OFT) is a method to qualitatively and quantitatively measure general locomotor activity and willingness to explore in rodents. The hAPP transgenic models display a disinhibition-like phenotype in the elevated-plus maze and hyperactivity in arenas, such as the open field, and so the open field test was used to test behavior of APP695 mice compared to WT mice (FIG. 18A). Compared with WT mice, the APP695 mice (AD) spent more time in the central zone (Zone 2+Zone 3) and less time in the peripheral zone of the field (FIG. 18A) and showed an increased frequency of total mobility (p<0.05; FIG. 16B). The behavior of the co-immunized and Aβ42 protein immunized mice resembled WT littermates, with more time spent in the periphery and less in the center. Likewise, the frequency of total mobility was significantly decreased compared with the untreated AD mice (FIG. 18A, FIG. 18B). The DNA-immunized group also showed some improvements, but the differences were not significant when compared with the co-immunized and Aβ42 protein immunized groups.

### Example 15

### Co-Immunization of Aβ42 Improves the Learning and Memory of APP695 Mice in MWM Tests

To assess spatial learning and memory functions, all animals underwent MWM tests 14 days after the last immunization. As shown in FIG. 18C, there was a significant difference overall in escape latency amongst the four groups (group effect: F(4,36)=11.13, p<0.01; training day effect: F(4,144)=12.45, p<0.01; group×training day interaction: F(12,144)=1.087, p>0.05). The latency to find the submerged platform decreased every day in each group, but the escape latency in the untreated APP695 mice was significantly longer than that of the WT group (p<0.01). The co-immunized and protein immunized AD mice showed significantly decreased escape latencies compared with the untreated APP695 controls (p<0.05), almost down to WT levels. The DNA-immunized mice also improved their learning behavior, but not significantly compared to co-immunization and Aβ42 protein immunized groups.

In a probe test, the frequency of crossing the platform was measured for 60 sec on the 4th day after the last acquisition test. As shown in FIG. 18D, there was an overall significant difference in the frequency of crossing the platform amongst the five groups (F(4,36)=10.45, p<0.01). The latency was decreased in the untreated APP695 mice compared with WT mice (p<0.01). Duration times in the platform zone were significantly increased (p<0.01) from both co-immunized and protein immunized groups compared with the untreated APP695 mice, whereas the DNA-immunized group showed no improvements compared to AD mice (p>0.05).

Taken together, these results showed that Aβ42 protein immunization as well as the co-immunization efficiently ameliorated cognitive deficits in AD mice.

### Example 16

### Co-Immunization Affects Inflammatory Cytokines in Brain

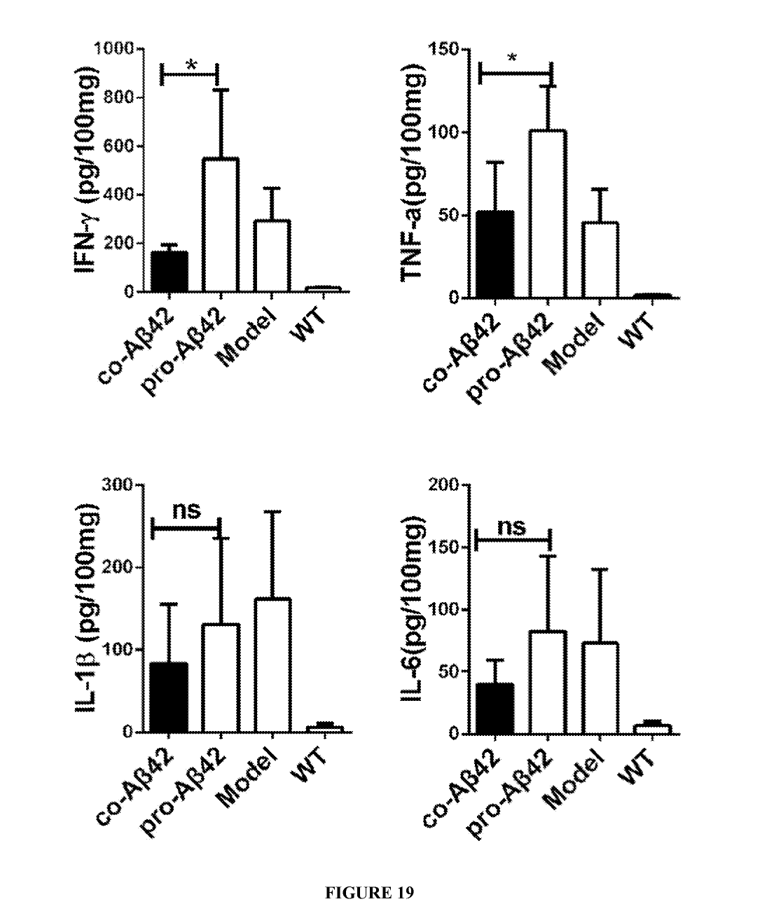

Inflammatory cytokines in brain are believed to induce brain inflammation and, in severe cases, meningoencephalitis. To compare the level of cytokines in brains of immunized and control groups, brain tissues were homogenized and assayed for cytokine expressions by CBA (Cytometric Beads Array) (FIG. 19). The results showed that APP695 mice had an elevated basal inflammatory state in the brain when compared to WT mice, indicated by increased expression of IFN-γ, TNF-α, IL-1β, and IL-6. Aβ42 protein vaccination induced a further increase in IFN-γ and TNF-α levels compared to untreated APP695 mice. In contrast, in the co-immunization group cytokine levels were similar to untreated APP695 mice (FIG. 19, TNF-α) or even lower (IFN-γ, IL-1β, and IL-6). These results suggested that the co-immunization strategy could induce anti-inflammatory effects in vivo by down-regulating expression of IFN-γ and TNF-α.

### Example 17

### Co-Immunization Suppresses T Cell Proliferation by Inducing CD25− iTreg Cells

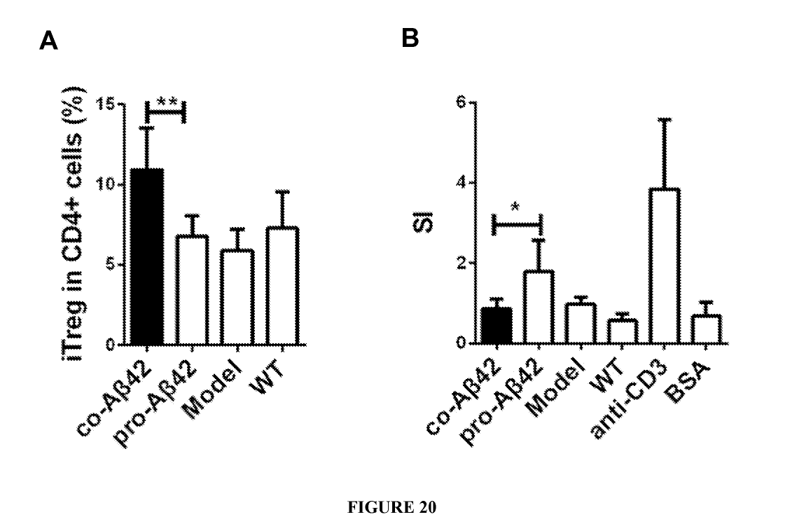

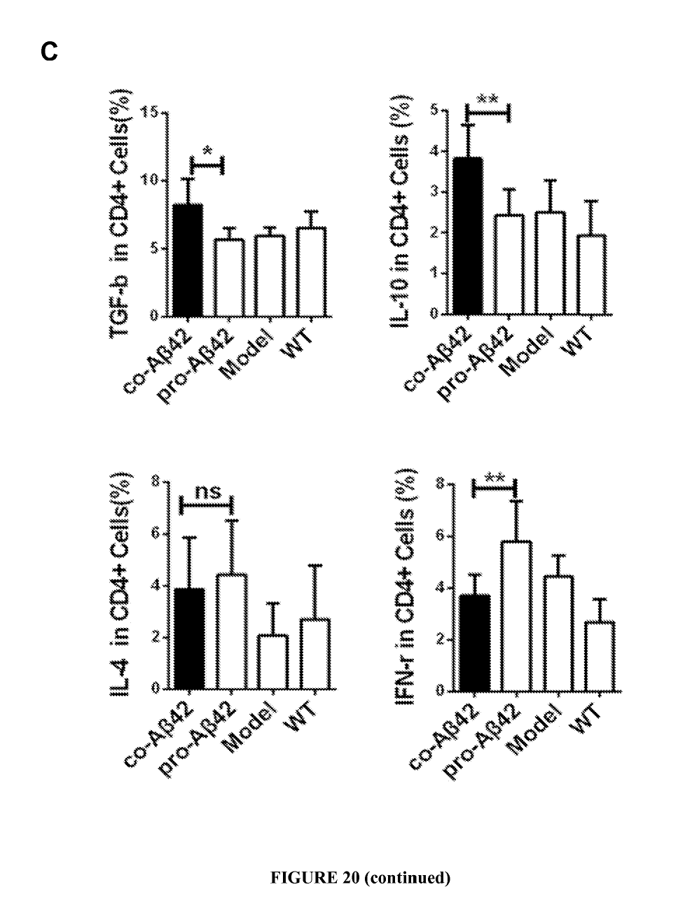

It was previously demonstrated that co-immunization induced high titers of IgG against protein antigen. Co-immunization also induced antigen-specific iTreg cells to suppress CD4 T cell responses in an antigen specific manner. To test whether this would also hold true for anti-Aβ42 co-immunization, a T cell proliferation assay was completed 7 days after the fifth immunization (after the behavior tests had been completed). Aβ42 protein immunization induced a 2-fold stimulation compared to control-immunized mice, whereas the co-immunization did not induce any T-cell proliferation (FIG. 20A). The suppression of T-cell responses was apparently associated with an increase of the level of CD25− iTreg in the co-immunized mice (FIG. 20B) in a dose dependent manner (FIG. 23).

It was next determined if the induced iTreg affected T cell production of inflammatory cytokines. After immunization, CD4 T cells were isolated and intracellular staining was performed. As shown in FIG. 20C, the level of IFN-γ was down regulated, but levels of Foxp3, IL-10, and TGF-α were upregulated by the co-immunization regimen when compared with the Aβ42 protein immunized group. In contrast, no effect on the level of IL-4 was observed (FIG. 20C). These results suggested that the co-immunization protocol induced a Th1 suppressive response but did not affect the humoral response.

### Example 18

### Co-Immunization Prevents Induced Experimental Autoimmune Encephalomyelitis

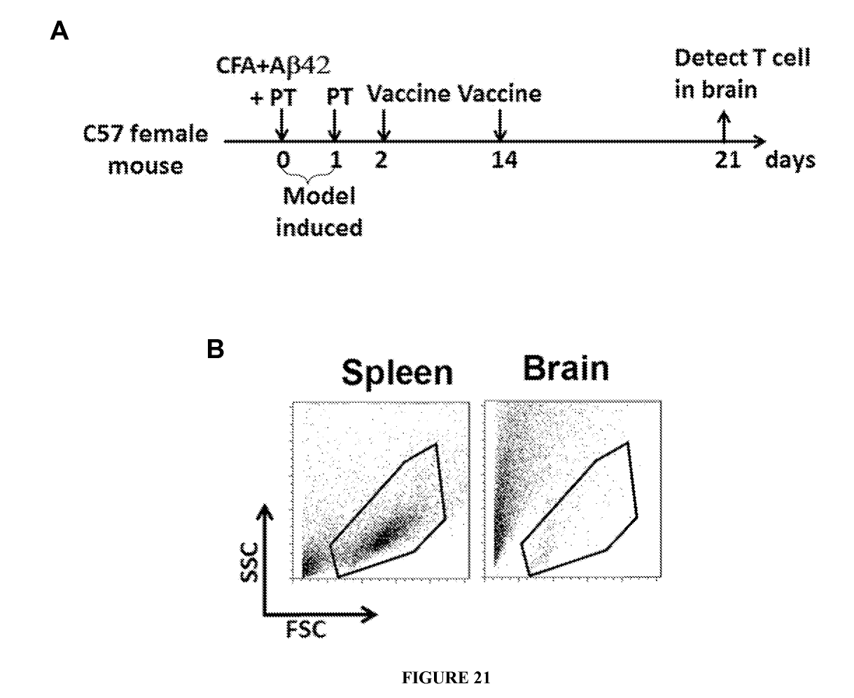

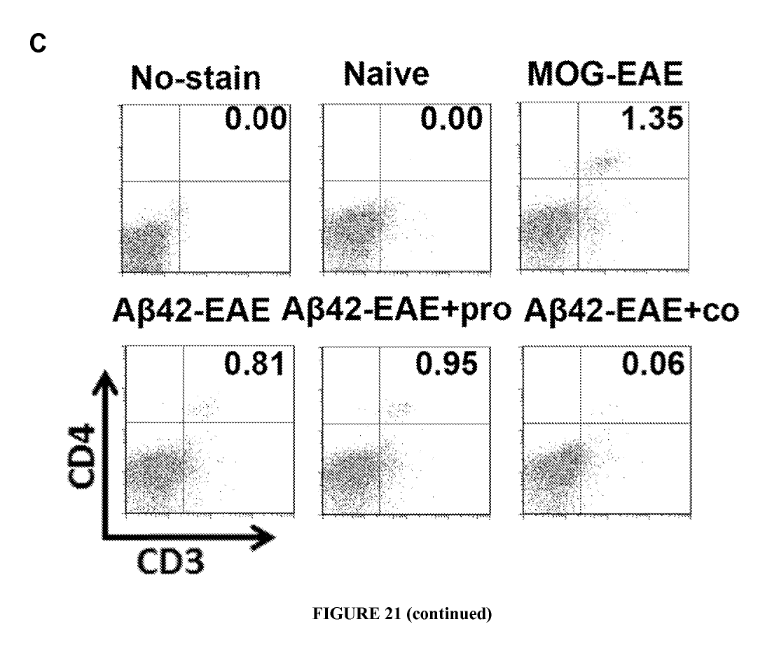

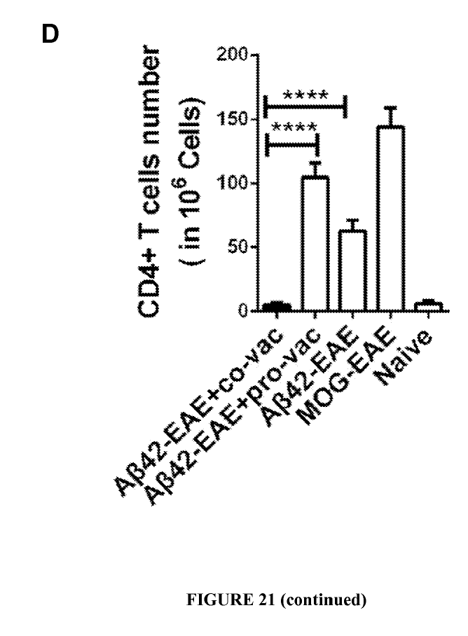

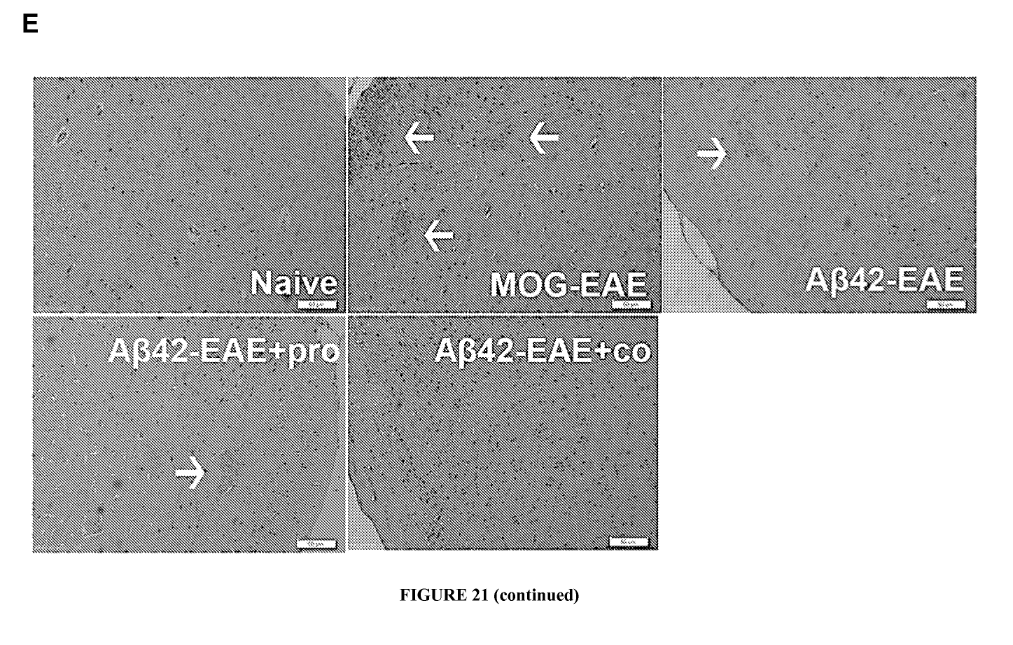

Since previous protein vaccinations caused brain inflammation leading to meningoencephalitis, the vaccines were examined for their effect on inflammation and meningoencephalitis. EAE presents an excellent model for T-cell-mediated autoimmune disease in brain and mimics the Aβ42 vaccine-induced meningoencephalitis. To test the efficacy of the co-immunization strategy in preventing EAE, an EAE animal model was first established by immunizing with (on day 0) either MOG35-55 peptides+CFA+PT (MOG-EAE model) or with Aβ42 protein+CFA+PT (Aβ42-EAE model) and then with PT on day 1. The EAE-induced mice were further co-immunized or protein immunized on days 2 and 14. A scheme of the strategy is depicted in FIG. 21A. T cells infiltrating the brain were isolated and analyzed by flow cytometry on day 21 after staining the single cell suspensions with anti-CD3 and anti-CD4 (FIG. 21B). A large number of CD3+ and CD4+ T cells had infiltrated brains of MOG-induced or Aβ42-induced EAE mice, potentially causing meningoencephalitis. Even larger brain infiltrations were observed in the Aβ42-induced EAE mice after subsequent Aβ42 protein immunization (FIG. 21C, FIG. 21D). However, in sharp contrast, the number of T cells was dramatically reduced to the level of the naive group when the mice were co-immunized (FIG. 21C and FIG. 21D; P<0.0001). These results were confirmed in a histopathological analysis of H&E stained brain sections. MOG-EAE and Aβ42-EAE caused large clusters of infiltrated T-cells (FIG. 21E), whereas in the co-immunized animals no T cell infiltration was detected and resembled the situation in naïve mice (FIG. 21E). This result demonstrated that co-immunization with Aβ42 DNA and Aβ42 protein induced a protective response against brain inflammation.

### Example 19

### Co-Immunization-Induced iTreg Suppressed Brain Inflammation

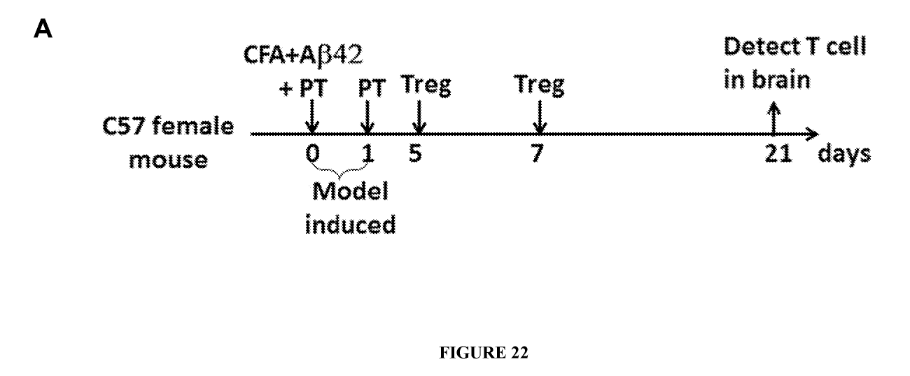

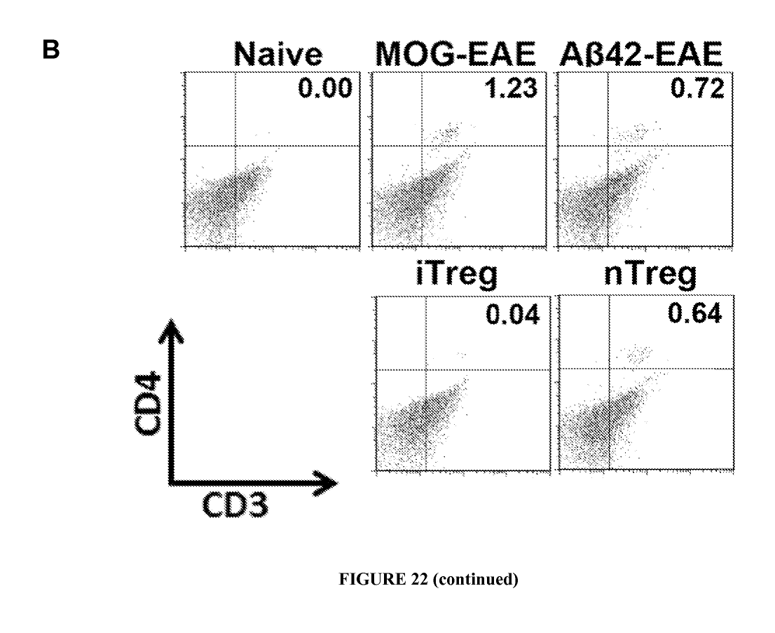

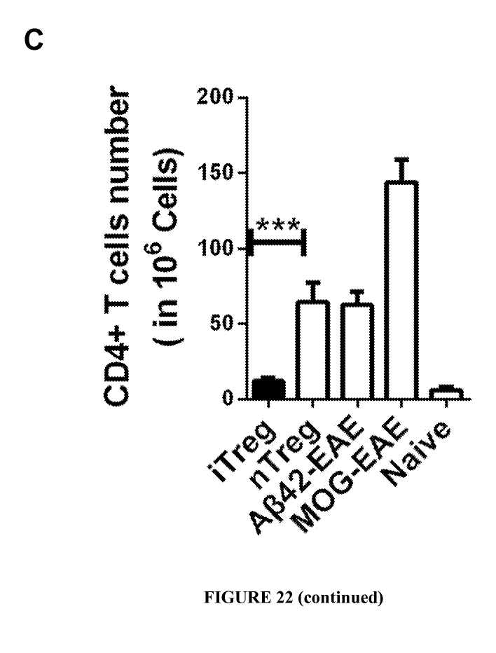

It was examined whether the iTreg acted as the main suppressor of Aβ42 antigen specific T cells. To this end, iTreg cells were isolated from co-immunized Foxp3-eGFP mice and adoptively transferred into Aβ42-induced EAE model animals as recipients on days 5 and 12 after EAE induction (FIG. 22A). As a control, nTregs were transferred. T cells isolated from each brain were analyzed by FACS (FIG. 22B) and the sum of results is presented as a bar graph in FIG. 22C. On average, more than one hundred T cells per 10⁶ brain cells infiltrated brains of MOG-induced EAE mice, and about 70 T cells per 10⁶ brain cells infiltrated Aβ42-induced EAE mice. Transferring nTreg had no impact on T-cell infiltration, but transfer of iTreg significantly reduced T cell infiltration in brains of Aβ42-induced EAE mice (p<0.001; FIG. 22C). This result demonstrated that the iTreg cells induced by the co-immunization did indeed suppress effector T cell infiltration into the brain.

In conclusion, these data demonstrated that the co-immunization regimens with Aβ42 protein and Aβ42 coding DNA induced iTregs that strongly reduced brain inflammation and infiltrations of T cells into brains. At the same time, a robust anti-Aβ42 antibody response was elicited that effectively reduced plaque formation. Therefore co-immunization is an attractive and novel strategy in AD vaccine development.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, can be made without departing from the spirit and scope thereof.

1. A vaccine comprising an amyloid beta antigen and a nucleic acid encoding the amyloid beta antigen, or a variant thereof.

2. The vaccine of claim 1, wherein the amyloid beta antigen comprises one or more amyloid beta polypeptides in tandem.

3. The vaccine of claim 2, wherein the amyloid beta polypeptide comprises an amino acid sequence selected from the group consisting of SEQ ID NO: 1, an amino acid sequence that is 95% identical over the entire length of the amino acid sequence of SEQ ID NO: 1, a fragment of SEQ ID NO: 1 comprising at least 20 amino acids, and an amino acid sequence that is 95% identical to a fragment of SEQ ID NO: 1 comprising at least 20 amino acids.

4. The vaccine of claim 2, wherein the amyloid beta antigen comprises two copies of the amyloid beta polypeptide in tandem.

5. The vaccine of claim 4, wherein the nucleic acid encodes an amyloid beta antigen variant comprising one copy of the amyloid beta polypeptide.

6. The vaccine of claim 2, wherein the amyloid beta antigen comprises one copy of the amyloid beta polypeptide.

7. The vaccine of claim 6, wherein the nucleic acid encodes an amyloid beta antigen variant comprising two copies of the amyloid beta polypeptide in tandem.

8. The vaccine of claim 3, wherein the amyloid beta polypeptide is Aβ(42).

9. The vaccine of claim 1, wherein the nucleic acid further comprises an expression vector.

10. The vaccine of claim 9, wherein the expression vector is operably linked to regulatory elements.

11. The vaccine of claim 10, wherein the regulatory elements are functional in a human cell.

12. The vaccine of claim 8, wherein the expression vector is a plasmid.

13. A method for preventing or treating Alzheimer's Disease in a subject, the method comprising administering to the subject the vaccine of claim 1.

14. The method of claim 13, wherein T cell proliferation is suppressed in the subject relative to a control.

15. The method of claim 13, wherein the level of iTreg cells is increased relative to a control.

16. The method of claim 13, wherein infiltration of CD4+ T cells into the brain is suppressed in the subject relative to administration of amyloid beta antigen alone.

17. The method of claim 13, wherein the levels of IFN-γ are decreased relative to a control and the levels of Foxp3, IL-10, and TGF-β are increased relative to a control.

18. The method of claim 13, wherein the level of IgG is increased in the subject relative to a reference level.

19. The method of claim 13, wherein the percent area of the subject's brain comprising amyloid beta plaque is reduced to 0-0.3%.

20. The method of claim 13, further comprising testing for spatial learning and memory.

21. The method of claim 20, wherein spatial learning and memory is tested with Morris Water Maze.

22. The method of claim 21, wherein spatial learning and memory is improved by 10-50%.

23. A method for reducing or slowing the rate of formation of amyloid beta plaques in a subject, the method comprising administering to the subject the vaccine of claim 1.

24. A method for treating encephalitis in a subject, the method comprising administering to the subject the vaccine of claim 1.

25. A method of reducing brain swelling in a subject, the method comprising administering to the subject the vaccine of claim 1.
